SEQUENCE LISTING (4 sequences)

SEQ ID NO: 1 - protein, 293 aa, Danio rerio
MAVVHAEGNVWIKQWDHRFHMYGGQTCSPLMAGSWDDRTPLHDAALQGRLLPLRRLLSQGYNVGMATLDGITALHEACVGGHFTCAKLLLEHGADANAVTFDGATPLFSACCSGNPALVSLILTHSSAHHPAHLLCSPLHEAAKRGHTACVELLLSHGVNVDMELPSVGTALYCACEVKSTDCVLTLLILGADVQCGRGLDTPLHAACRVGGAKEAELLLEHGADRTSRNSEGKTPLDLTSDQSIKHLLQTAGTCSLSQLCRWCIRRSLGQKGLNKTKTLCLPHMLHNYLLYH

SEQ ID NO: 2 - DNA, 879 bp, Danio rerio
atggccgtggttcatgctgaagggaatgtctggattaagcaatgggatcacaggtttcacatgtatggaggacaaacatgtagtccactaatggcaggctcctgggacgacagaacacctttacacgatgctgctttgcaaggaagactgcttccactgagaagactcctctcacagggctacaatgttggaatggcgactttagatggaatcacagcactgcatgaagcttgtgttggaggacatttcacctgtgctaaacttctcctggaacatggtgcagatgcaaatgcagtgacttttgatggagccactcctctgttcagtgcctgctgcagtggaaaccccgcccttgtcagcctcattttgacacacagctccgcccaccatccagctcacctgctctgctcacctctgcatgaagctgcaaagagaggtcacacggcctgtgttgaactgctgttgtctcatggtgtgaacgtggacatggagctgcccagtgttggaacagcgctgtactgtgcatgtgaagtcaagagcacagactgtgtactgaccctgctgatcttaggtgctgatgtacaatgtgggcgtggccttgacacacctttacatgctgcatgcagagttggtggagcaaaagaggcggagctactattagaacacggggctgatcgtacatctagaaactctgagggaaagacacctctggatctgacctcagatcagagcatcaaacacctcttgcagactgcaggtacctgctctctgtctcagctatgcagatggtgcattcgacgctcactgggacaaaaaggactcaacaaaaccaagaccctttgcttaccacatatgctgcacaattatcttctctatcat

SEQ ID NO: 3 - protein, 329 aa, Homo sapiens
MSVLEENRPFAQQLSNVYFTILSLFCFKLFVKISLAILSHFYIVKGNRKEAARIAAEFYGVTQGQGSWADRSPLHEAASQGRLLALRTLLSQGYNVNAVTLDHVTPLHEACLGDHVACARTLLEAGANVNAITIDGVTPLFNACSQGSPSCAELLLEYGAKAQLESCLPSPTHEAASKGHHECLDILISWGIDVDQEIPHLGTPLYVACMSQQFHCIWKLLYAGADVQKGKYWDTPLHAAAQQSSTEIVNLLLEFGADINAKNTELLRPIDVATSSSMVERILLQHEATPSSLYQLCRLCIRSYIGKPRLHLIPQLQLPTLLKNFLQYR

SEQ ID NO: 4 - DNA, 990 bp, Homo sapiens
atgtcggtgttagaagaaaatcggccgtttgctcaacaattatccaatgtctactttacaatactttcgctgttctgttttaagctttttgtgaaaatcagccttgccatcctcagtcatttctacatagtgaaaggcaaccgcaaggaagcggcaaggatagcagctgaattttatggagtaacccaaggacaaggttcctgggcagatcgatcaccactacatgaagcagcaagtcaaggtcgccttcttgctctgagaacattattatcacagggttataatgtaaatgcagtaaccttagaccatgtcaccccattgcacgaagcctgccttggagatcacgtggcatgtgccagaactctgctggaagcaggagctaatgtaaatgcaatcacgatagatggcgtgactccgttattcaacgcatgctcccaaggcagtccaagctgtgcagagctgcttctggagtatggtgccaaagcccagctggagtcatgtcttccatccccaacgcatgaggccgccagtaaaggtcaccatgaatgtcttgacatcctgatatcctggggcatagatgttgaccaagaaattcctcatttgggaactcctctctatgtagcttgtatgtcacagcaattccattgcatctggaagcttctttatgctggtgctgacgtacagaaaggcaaatattgggatactccattacatgctgctgctcaacaatccagcacagaaattgtaaacttactgctagaatttggagcagatatcaatgccaaaaatacagagcttctgcgacctatagatgtagctacgtctagcagtatggtggaaagaatattgcttcaacatgaagctaccccaagctctctttaccaactttgccgactctgtatccgaagctacataggaaaaccaagattgcaccttatcccacaactccagctgccaacgttactgaagaatttcttacagtatcgataa

(19) United States
(12) Patent Application Publication
(10) Pub. No.: US 2006/0127371 A1
(43) Pub. Date: Jun. 15, 2006

(54) ASB transcription repressor proteins and nucleic acids and their application in expansion of stem cells

(76) Inventors: Maikel Petrus Peppelenbosch, Utrecht (NL); Danica Zivkovic, Utrecht (NL); Sander Diks, S-Hertogenbosch (NL); Robert Jozef Bink, Don Haag (NL)

Correspondence Address: MCKENNA LONG & ALDRIDGE LLP, 1900 K STREET, NW, WASHINGTON, DC, 20006, US

(21) Appl. No.: 10/536,491

(22) PCT Filed: Nov. 25, 2003
(86) PCT No.: PCT/NL03/00931

§ 371 Date: Jan. 19, 2006

(30) Foreign Application Priority Data
Nov. 25, 2002 (NL) 02079903.7

(51) Int. Cl. A61K 35/30; C07H 21/04; C12P 21/06; C07K 14/705; C07K 14/16; C12N 5/08

(52) U.S. Cl. 424/93.7; 435/69.1; 435/368; 435/320.1; 530/350; 536/23.5

(57) ABSTRACT

The present invention relates to methods for expansion of stem or progenitor cells. These methods rely on Asb-a polypeptides or nucleic acids to temporarily suppress differentiation of the cells, thus allowing proliferation and self-renewal of the stem or progenitor cells. Abs-a polypeptides and coding sequences define a class of polypeptides and nucleic acids that are both structurally and functionally highly conserved among vertebrates. Asb-a polypeptides contain 6 ankyrin repeats and a SOCS box that mediate the effect of the polypeptide on the regulation of specific subsets of genes involved in differentiation. The invention discloses various methods to increase the intracellular concentration of an Asb-a polypeptide for suppression of terminal differentiation of the stem or progenitor cells. The invention further relates to Asb-a polypeptides and nucleic acids, vectors and host cells for use in methods for their production and for use in the method for expansion of stem or progenitor cells, as well as to stem or progenitor cells containing exogenous Asb-a polypeptides and nucleic acids.

### FIELD OF THE INVENTION

The present invention relates to methods for the expansion of stem or progenitor cells. In these methods Asb-a polypeptides and nucleic acids may be used to temporarily suppress differentiation of the cells, thus allowing the cells to proliferate and self-renew.

### BACKGROUND OF THE INVENTION

Normal development depends on tight control of cellular decisions to either proliferate, arrest growth, differentiate or initiate apoptosis. These decisions require a tight control of the transcription of relevant genes. For example, the development of the vertebrate central nervous system (CNS), i.e. proper morphogenesis and cell fate acquisition during neural induction, neurulation and neurogenesis, requires a fine-tuned balance between proliferation and differentiation.

Among the principal molecular mechanisms governing cell fate acquisition is the Delta-Notch pathway, in which the Notch transmembrane receptor activates target genes upon binding to the Delta ligand, leading to the amplification and consolidation of molecular differences between neighbouring cells (Artavanis-Tsakonas et al., 1999; Mumm and Kopan, 2000; Anderson et al., 2001). Upon ligand binding, the intracellular domain of Notch (Notch IC) is proteolytically cleaved and the thus-released Notch IC has been shown to be responsible for transmission of the Notch signal and can act as a constitutively active component to activate transcription (Kidd et al., 1998; Lecourtois and Schweisguth, 1998; Lieber et al., 1993; Struhl et al., 1993). Notch/Delta signalling is an evolutionary conserved mechanism that controls acquisition of a cell fate through cell-cell interactions (Artavanis-Tsakonas et al., 1999; Mumm and Kopan, 2000; Weinmaster, 2000). The output of Notch signalling is context dependent as, in interaction with other molecular pathways, it also regulates differentiation, proliferation and apoptosis.

Notch-Delta signalling operates between non-equipotential cells with an extrinsic or intrinsic molecular bias that modulates the signal and promotes a particular cell fate. Genes such as mastermind in Drosophila (Schuldt and Brand, 1999) and Nrarp in vertebrates (Krebs et al., 2001; Lamar et al., 2001) appear to modulate Notch/Delta signalling through binding and regulation of Notch IC and fit the category of intrinsic regulators. Zebrafish neurogenic or proneural mutants such as mind bomb (mib) and narrowminded (nrd), respectively, appear to fall within this category (Haddon et al., 1998a; Artinger et al., 1999).

Ubiquitination of specific proteins is an important regulatory mechanism to obtain fine-tuned signal transduction. Recently identified genes support a role for ubiquitination as an intrinsic mechanism that can rapidly control the activity of the Notch pathway. The Drosophila gene neuralised (neur) and its Xenopus homologue Xneur appear to be ubiquitin ligases that promote degradation of Delta thereby positively regulating Notch signalling (Deblandre et al., 2001; Lai et al., 2001). Drosophila Suppressor of Deltex Su(D), an E3 class ubiquitin ligase, negatively regulates Notch signalling by dominantly suppressing its positive regulator Deltex (Comell et al., 1999). Ubiquitin-dependent proteolysis of Notch components represents a fast and timely mechanism to regulate Notch signalling.

The SOCS box-containing proteins form a class of molecules that mediate ubiquitination of proteins thus targeting them for degradation by the proteasome (Zhang et al., 1999; De Sepulveda et al., 2000; Kile et. al., 2002). The SOCS box is a conserved carboxy-terminal motif termed “suppressor of cytokine signalling (SOCS) box” that was initially identified in the SOCS family of proteins (Hilton et al., 1998). The SOCS box couples SOCS proteins and their bound interacting partners with the Elongin BC complex that in turn binds to a putative E3 ubiquitin ligase. Thus SOCS box-containing proteins may act as adapters that bring ubiquitin ligases in contact with signalling proteins that would then be targeted for ubiquitination and degradation. The ASBs form a class of SOCS box-containing genes with multiple ankyrin repeats N-terminal of the SOCS box (Hilton et al., 1998, Kile et al., 2000). However, little is known about the physiological role of ASB genes, although it has been suggested that they may be involved in differentiation and/or proliferation (Kohroki et al., 2001, Guibal et al., 2002).

For many biomedical purposes, there is an increasing interest in being able to expand stem and progenitor cells in culture. However, it has not proven simple to maintain cell viability for the stem cells and at the same time to ensure that the stem cells increase in numbers without losing their distinctive phenotype of pluri- or toti-potency. Current protocols for the in vitro culture of hematopoietic stem cells e.g. generally require one or a cocktail of cytokines, such as c-kit ligand (stem cell growth factor), fit-3, thrombopoietin, IL-6, and others. While a substantial increase in cell number can be obtained with such cultures, they do not provide for expanded number of cells that retain a capacity for long term repopulation of all hematopoietic lineages (see Domen and Weissman (1999) Mol. Med. Today 5: 201-8; or Ziegler and Kanz (1998) Curr. Opin. Hematol. 5: 434-40).

Stem cells have also been grown in co-culture with stromal cells. However, it is particularly desirable to expand stem cells in a culture of known composition, rather than relying upon the presence of other cells for their maintenance.

U.S. Pat. No. 6,465,249 discloses expansion of murine hematopoietic stem cells in vitro by increasing the levels of β-catenin in the cell. The expanded cells substantially maintain their original phenotype including the ability to give rise to multiple types of differentiated cells. The intracellular levels of β-catenin are manipulated by introduction into the cell of a genetic construct encoding β-catenin. The long term cell culture medium substantially lacks stromal cells and cytokines.

However, there continues to be a need for methods for expansion of stem cells and progenitor cells that are also applicable to stem and progenitor cells of lineages other than the hematopoietic lineage. Furthermore, there continues to be a strong demand for improvements in the in vitro culture of stem cells and progenitor cells in general. The present invention addresses these needs.

### DESCRIPTION OF THE INVENTION

The present invention finds a basis in the discovery that in zebrafish, the ankyrin and SOCS box (ASB) containing gene, asb-a, has been identified as a gene that is expressed in the Delta-Notch synexpression group and that interferes with cell decisions mediated by the Delta-Notch pathway (see Examples herein). Asb-a is structurally related to Notch IC as both proteins contain six ankyrin repeats. In addition, Asb-a contains a C-terminal SOCS box and can homodimerise as well heterodimerise with Notch1a IC and Nrarp, a negative regulator of Notch signalling (Lamar et al., 2001). In vivo gain-of-function assays demonstrate that upon overexpression, Asb-a interferes with neurogenesis and somitogenesis and affects the expression of zebrafish hairy/Enhancer of split related gene, her1, that is a genetic target of Notch (Takke et al., 1999). Loss-of-function experiments demonstrate that one aspect of asb-a is that it can behave as a neurogenic gene, as neurogenin1 (ngn1), a proneural gene, that is upregulated followed by an increased number of neurons developing in the neural tube (see Examples herein).

As a member of the class of ASB genes, the zebrafish Asb-a contains a SOCS box. Four classes of SOCS box containing proteins have been identified: the SOCS family that share an SH2 domain, the WSB family that share a WD40 domain, the SSB family that share a SPRY domain and the ASB family that share ankyrin repeats (Hilton et al., 1998). The SOCS-SH2 family of proteins appear to function in a negative feedback loop in the cytokine induced JAK/STAT signalling pathway by inhibiting JAK kinases (Endo et al., 1997; Naka et al., 1997; Starr et al., 1997) or through interactions with receptor tyrosine kinases (Yoshimura et al., 1995; Matsumoto et al., 1997).

Interestingly overexpression of the zebrafish Asb-a has important consequences for NGF-induced neuronal differentiation of neurogenic stem cells like PC12 cells. Overexpression of Asb-a in PC12 cells leads to a loss of NGF-induced inhibition of proliferation and terminal differentiation, superseding the effects resulting from Notch IC overexpression. The effects of Asb-a on neuronal differentiation in vivo appear to be cell-autonomous Remarkably, NGF treatment of PC12 cells transfected with Asb-a did not inhibit NGF signalling per se, but stopped further differentiation once the cells reached the neuronal precursor stage, as assessed by GAP43 expression. Overexpression of Asb-a may thus be used to expand neuronal precursor stem cells while supressing terminal differentiation.

The observation that Asb-a expression modulates rather than completely blocks NGF-induced differentiation indicates that Asb-a overexpression does not lead to a complete shutdown of NGF-induced signal transduction, but only allows certain signals emanating from the activated NGF receptor to reach the nucleus. In agreement, we observed that Asb-a overexpression impairs transcriptional activation by specific signal transduction pathways, for instance hardly affecting transactivation of c-myc RE and SRE containing reporter constructs (unpublished observations), but strongly interfering with the transactivation of κB enhancer-, CRE-, and ISRE-containing promoters, whereas also a variety of other promoters was moderately affected (unpublished observations). The fact that Asb-a inhibits other reporters than ISRE-mediated transactivation indicates that Asb-a may be a fairly unique SOCS box containing protein in that it not only inhibits Jak/Stat signalling but a variety of other signalling pathways as well. The transcriptional repression of specific signalling pathways exerted by Asb-a, is instrumental in the effects of Asb-a on cell fate.

SOCS box containing proteins mediate degradation of specific targets via ubiquitination (De Sepulveda et al., 2000; Kamura et al., 1998; Kamizono et al., 2001; Zhang et al., 2001; Kile et al., 2002). In addition, the SOCS box can bind the elongin B/C complex, which is part of an E3 ubiquitin ligase complex (Zhang et al., 1999; Kamura et al., 1998). We show in the Examples that, following stimulation with IL-1β, Asb-a overexpression led to the ubiquitination of IκBα and p65 immunoprecipitates and that this effect is dependent on the presence of the SOCS box. Importantly, we show a direct association of Asb-a with IκBα by immunoprecipitation and this interaction appears to be mediated by the six ankyrin repeats present in both proteins as similar binding of Asb-a to Notch1a IC and Nrarp appeared to be mediated by ankyrin repeats (see Examples). The ankyrin repeats in Asb-a thus mediate association of this protein with specific signal transduction mediating complexes, followed by SOCS box-dependent recruitment of ubiquitin ligases and subsequent destruction of the proteins involved. In this fashion, Asb-a actively inhibits signalling through specific signal transduction pathways.

The fact that Asb-a overexpression also interferes with transactivation of transcription factors that do not contain ankyrin repeats, like CREB or STAT, means that Asb-a can interact with still unknown proteins that regulate these pathways and these proteins may contain ankyrin repeats but not necessarily. Asb-a is thus recruited to transcription factors through intermediate proteins, capable of interacting with transcription factors and containing ankyrin repeats. In principle any ankyrin-repeat containing protein could act as bridge between a transcription factor and Asb-a thus allowing ubiquitination of the transcription factors involved. Various ankyrin repeat containing proteins capable of interacting with e.g. CREB or STATs, that are capable of such a function have been described (e.g. D'Amico et al., 2000; Kamura et al., 1998).

The fact that a zebrafish Asb-a-protein this protein remains active in mammalian cells, and even in human cells, demonstrates its evolutionary conserved regulatory mechanism. Indeed, we identified 3 mammalian Asb-a homologues, ASB-5, -9 and -11 (see Examples). These proteins share more homology with each other than with Asb-a, but share more homology with Asb-a than with other ASB proteins and thus Asb-a, ASB5, ASB9 and ASB11 and homologues from other vertebrates, likely share a common ancestral gene (see definition of Asb-a polypeptides and nucleic acids below) and a common function. In the various applications of the invention Asb-a homologues may thus be substituted for each other.

Expression of most (but not all) ASB proteins is tissue specific (Kile et al., 2000). For instance expression of ASB5 is at least muscle specific since most of the known ESTs that come up when searched for ASB5 are derived from muscle or myotubule cell line and is supported by cell specific localised expression of asb-a in zebrafish embryos. This indicates that the human ASB-5, -9, and -11, have a similar action as Asb-a and act in a tissue specific manner by means of a tissue specific expression pattern of these genes during organogenesis. The function of these proteins thus is the regulation of proliferation of specific precursor cell populations and to establish the final size of the various compartments (e.g. muscle and nervous tissue). This allows to use these proteins and the encoding nucleotide sequences inter alia for the ex vivo expansion of these and other precursor cell populations.

In a first aspect the invention therefore relates to a method for in vitro expansion of mammalian stem cells. The method preferably comprises the steps of (a) providing an Asb-a polypeptide to the stem cells whereby the intracellular concentration of the Asb-a polypeptide is sufficient to prevent differentiation of the cells; and, (b) culturing the stem cells for a period of time sufficient for the cells to divide and self-renew.

An Asb-a polypeptide is herein defined as a polypeptide having an amino acid sequence with at least 39, 39.5, 40, 40, 7 or 43.7% amino acid identity with SEQ ID NO: 1 or 3. A non-mammalian vertebrate Asb-a polypeptide is preferably defined as a polypeptide having an amino acid sequence with at least 45, 50, 60, 70, 80 or 90% amino acid identity with SEQ ID NO: 1. A mammalian Asb-a polypeptide is preferably defined as a polypeptide having an amino acid sequence with at least 45, 50, 60, 70, 80 or 90% amino acid identity with SEQ ID NO: 3.

An Asb-a polypeptide may further be defined in that it preferably has 6 ankyrin repeats (see Lux et al., 1990). An ankyrin repeat is defined as an amino acid sequence of about 33 amino acids in which at least 5 amino acids of the ankyrin consensus sequence are present, whereby the ankyrin consensus sequence is defined as: X G X T P L H X A A X X G H X X X V/A X X L L X X G A X X N/D X X X X; whereby X may be any amino acid. An Asb-a polypeptide may further be defined in that it preferably has a SOCS box (see Hilton et al., 1998), which is preferably located at or near the C-terminal end of the Asb-a polypeptide. A SOCS box is defined as an amino acid sequence of about 40 amino acids in which at least 15 amino acids of the SOCS box consensus sequence are present, whereby the SOCS box consensus sequence is defined as: Φ/P X P/T/S L Q Y/H Φ C R X X Φ X X X Φ X X X X X X X X Φ X L P Φ P X X F/Y L X F/Y X; whereby Φ=A, F, I, L, M, S, T or V; and whereby X may be any amino acid. Preferred Asb-a polypeptides are the zebrafish Asb-a and the human and murine ASB-5, ASB-9 and ASB-11 polypeptides.

An Asb-a polypeptide may further be functionally defined in that it is preferably capable of homodimerisation, or heterodimerisation with Notch IC or with Nrarp, or association with IκB-α, as may be assayed as indicated in the Examples herein. An Asb-a polypeptide may further be functionally defined in that overexpression of an Asb-a polypeptide in PC12 cells suppresses NGF-induced terminal neuronal differentiation of these cells while allowing a conversion of the cells to a neuronal precursor state and allowing proliferation of these cells, as may be assayed as indicated in the Examples herein. The capability of an Asb-a polypeptide is merely used here to functionally define the polypeptide and is not intended to limit the method of the invention to only thid type of cells. An Asb-a polypeptide may further be functionally defined by assaying any of the other biological or biochemical activities of the polypeptide described in the Examples herein.

Included in the term Asb-a polypeptide are natural allelic variants of Asb-a as they may occur in vertebrates. Similarly included in the term Asb-a polypeptide are variants of naturally occuring Asb-a polypeptides with substitutions, insertions of deletions of one or more amino acids, while maintaining the above defined functions. Asb-a polypeptides may be employed in the methods of the invention and are in themselves another aspect of the invention.

As described below, also nucleic acids comprising a nucleotide sequence encoding an Asb-a polypeptide may be employed in the methods of the invention and are in themselves another aspect of the invention. A nucleotide sequence encoding an Asb-a polypeptide is herein defined as a nucleotide sequence encoding an Asb-a polypeptide as defined above. A nucleotide sequence encoding an Asb-a polypeptide preferably has at least 35, 40, 50, 60 or 70% identity with the nucleotide sequences of SEQ ID NO: 2 or 4. Alternatively a nucleotide sequence encoding an Asb-a polypeptide may be defined by its capability of hybridising to the nucleotide sequences of SEQ ID NO: 2 or 4 under moderate or stringent conditions as described below.

“Sequence identity” is herein defined as a relationship between two or more amino acid (polypeptide or protein) sequences or two or more nucleic acid (poly- or oligo-) nucleotide sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gin or his; Asp to glu; Cys to ser or ala; Gln to asn; Glu to asp; Gly to pro; His to asn or gin; Ile to leu or val; Leu to ile or val; Lys to arg; gin or glu; Met to leu or ile; Phe to met, leu or tyr; Ser to thr; Thr to ser; Trp to tyr; Tyr to trp or phe; and, Val to ile or leu.

Nucleic acid sequences encoding an Asb-a polypeptide as defined above may also be defined by their capability to hybridise with the (complementary strand of) the nucleotide sequences of SEQ ID NO: 2 or 4, preferably under moderate, or more preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions so as to specifically identify sequences varying in identity between 50% and 90%.

The nucleic acid molecules of the invention preferably encode an Asb-a polypeptide that is a vertebrate or a mammalian Asb-a polypeptide, preferably a human, mouse or zebrafish Asb-a polypeptide.

The term stem cell is used herein to refer to a mammalian cell that has the ability both to self-renew, and to generate differentiated progeny (see Morrison, 1997, Cell). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterised by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny. Stem cells of interest for use in the methods of the invention include neurogenic and neural crest stem cells (see Morrison et al. (1999) Cell 96:737-749) and progenitor cells derived therefrom. Stem cells of interest further include hematopoietic stem cells and progenitor cells derived therefrom (U.S. Pat. No. 5,061,620); embryonic stem cells as well as cells of non-embryonic origin, such as e.g. stem cells derived from a foetus, neonate or adult; mesenchymal stem cells (U.S. Pat. No. 6,387,367); mesodermal stem cells; endodermal stem cells; ectodermal stem cells.

Other hematopoietic “progenitor” cells of interest include cells dedicated to lymphoid lineages, e.g. immature T cell and B cell populations, monocytes, macrophages, dendritic cells, basophils, eosinophils, neutrophils, mast cells, natural killer cells. The methods of the present invention are useful in expanding selected populations of these cells.

Other neurogenic “progenitor” cells of interest include cells dedicated to central neuronal lineage and peripheral neuronal lineages, e.g. immature dopaminergic neurons, cholinergic neurons, GABAergic neurons, adrenergic neurons, noradrenergic neurons, glutaminergic neurons, neuropeptides-specific progenitor cells (e.g. vasopressin, oxytocin, vaso-intestinal peptide, corticotropin releasing hormone, prolactin, angiotensin, endorphin, somatostatin, substance P, bombesin, neurotensin, melonotropin, bradykinin, TRH, cholecystokinin) as well as neuroblasts, gliablasts, schwann cell progenitors, astrocyte progenitors, Purkinje cell progenitors. Other progenitor cells of interest include endothelial cells, cardiomyoblasts, cardiomyocytes, renal precursor cells, hepatic precursor cells, pancreatic precursor cells, as well as endocrine (precursor) cells (e.g. insulin secreting cells, adrenal stem cells, thyroid precursor cells, parathyroid precursor cells) and skin precursor cells.

Purified populations of stem or progenitor cells may be used to initiate the cultures. For example, neural crest stem cells may be positively selected with antibodies specific for low-affinity nerve growth factor receptor (LNGFR), and negatively selected for the markers sulfatide, glial fibrillary acidic protein (GFAP), myelin protein Pₒ, peripherin and neurofilament.human. Similarly, hematopoietic stem cells may be positively selected using antibodies specific for CD34, thy-1; or negatively selected using lineage specific markers which may include glycophorin A, CD3, CD24, CD16, CD14, CD38, CD45RA, CD36, CD2, CD19, CD56, CD66a, and CD66b; T cell specific markers, tumor specific markers, etc. Markers useful for the separation of mesodermal stem cells include Fc.gamma.RII, Fc.gamma.RIll, Thy-1, CD44, VLA-4.alpha., LFA-1.beta., HSA, ICAM-1, CD45, Aa4.1, Sea-1, etc. Human mesenchymal stem cells may be positively separated using the markers SH2, SH3 and SH4 (described in U.S. Pat. No. 5,486,359). Several methods to positively separate the desired stem or progenitor cells from other cells in the source material are available to the skilled person in the art, including e.g. fluorescence activated cell sorting or MACs.

The cells of interest are typically mammalian, where the term refers to any animal classified as a mammal, including humans and non-human mammals such as domestic and farm animals, and zoo, laboratory, sports, or pet animals, such as dogs, horses, cats, cows, mice, rats, rabbits, etc. Preferably, the mammal is human.

The cells which are employed may be fresh, frozen, or have been subject to prior culture. They may be fetal, neonate, adult. Neurocrest cells may be obtained from embryos and foetuses employing microsurgery and subsequent appropiate culture. Hematopoietic cells may be obtained from fetal liver, bone marrow, blood, particularly G-CSF or GM-CSF mobilised peripheral blood, or any other conventional source. The manner in which the stem cells are separated from other cells of the hematopoietic or other lineage is not critical to this invention. As described above, a substantially homogeneous population of stem or progenitor cells may be obtained by selective isolation of cells free of markers associated with differentiated cells, while displaying epitopic characteristics associated with the stem cells.

The stem or progenitor cells are grown in vitro in an appropriate liquid nutrient medium. Generally, the seeding level will be at least about 10 cells/ml, more usually at least about 100 cells/ml and generally not more than about 10⁵ cells/ml, usually not more than about 10⁴ cells/ml.

Various media are commercially available and may be used, including Ex vivo serum free medium; Dulbecco's Modified Eagle Medium (DMEM), RPMI, Iscove's medium, etc. The medium may be supplemented with serum or with defined additives. Appropriate antibiotics to prevent bacterial growth and other additives, such as pyruvate (0.1-5 mM), glutamine (0.5-5 mM), 2-mercaptoethanol (1-10. times. 10⁻⁵ M) may also be included.

Culture in serum-free medium is of particular interest. The medium may be any conventional culture medium, generally supplemented with additives such as iron-saturated transferrin, human serum albumin, soy bean lipids, linoleic acid, cholesterol, alpha thioglycerol, crystalline bovine hemin, etc., that allow for the growth of hematopoietic cells.

Preferably the expansion medium is free of cytokines, particularly cytokines that induce cellular differentiation. The term cytokine may include lymphokines, monokines and growth factors. Included among the cytokines are thrombopoietin (TPO); nerve growth factors such as e.g. NGF-β; platelet-growth factor; transforming growth factors (TGFs) such as e.g. TGFα- and TGF-β; erythropoietin (EPO); interferons such as e.g. interferon-α, -β, and -γ, colony stimulating factors (CSFs) such as e.g. macrophage-CSF (M-CSF); granulocyte-macrophage-CSF (GM-CSF); and granulocyte-CSF (G-CSF); interleukins (ILs) such as e.g. IL-1, IL-1α, IL-2, IL-3, IL-4, IL-5, IL-6, IL-7, IL-8, IL-9, IL-11, IL-12, IL-18. In some circumstances, proliferative factors that do not induce cellular differentiation may be included in the cultures, e.g. c-kit ligand, LIF, CNTF, aggregation protocols, DMSO treatment, retinoic acid, butyrate, Epidermal Growth Factor, Inhibins and Activins, Fibroblast growth factors (e.g. a-FGF, b-FGF, FGF-8, k-FGF), CNTF and other Neurotrophins, Netrins, Neuropilins and Repulsins, Slit and the like.

Preferably in the method of the invention, the intracellular concentration of the Asb-a polypeptide is maintained at a level sufficient to prevent differentation of the cells for a period of time sufficient for the cells to divide and self-renew until the population of the stem cells has reached a desired size. Thus, preferably the intracellular concentration of the Asb-a polypeptide is not permanently maintained at a level that to prevent differentation, but only temporalily such that after expansion to a desired cell population size the cell may be allowed or induced to differentiate into a desired phenotype. Various methods are available to only temporarily maintain the intracellular concentration of the Asb-a polypeptide at a level that prevents differentation.

For example, in one method, the Asb-a polypeptide is provided to the cells by addition of an exogenous Asb-a polypeptide to the culture medium. Exogenous Asb-a polypeptide is understood to mean that the Asb-a polypeptide is produced in other cells than the stem cells to be expanded. The Asb-a polypeptide may e.g. be synthesised by recombinant means in suitable host cells as described below. The exogenously added Asb-a polypeptide is taken up by the cells and its intracellular concentration decreases over time by natural turn-over and/or by dilution as a result of cell divisions. Further quantities of exogenous Asb-a polypeptide may be added to maintain the intracellular at a level that prevents differentiation until a desired cell population size is reached. To facilitate uptake of the exogenous Asb-a polypeptide, the polypeptide may be fused to a transport moiety. The Asb-a polypeptide may be genetically or chemically fused to a transport moiety. A preferred transport moiety is a fragment of an HIV tat protein, e.g. an HIV tat polypeptide that comprises the tat basic region amino acid sequence (amino acids 49-57 of naturally-occurring tat protein) but lacks the tat cysteine-rich region amino acid sequence (amino acids 22-36 of naturally-occurring tat protein) and lacks the tat exon 2-encoded carboxy-terminal domain (amino acids 73-86 of naturally-occurring tat protein). Alternatively, the uptake of the exogenous Asb-a polypeptide may be facilitated by incorporating the polypeptide into liposomes (see e.g. U.S. Pat. No. 6,245,427 and references cited therein).

In the method of the invention, the Asb-a polypeptide may also be provided to the cells by introducing an exogenous nucleic acid comprising a nucleotide sequence encoding the Asb-a polypeptide into the stem cells. The nucleic acid may be a DNA molecule that is transcribed into a mRNA that is translated to produce the Asb-a polypeptide, or the nucleic acid may an RNA molecule encoding the Asb-a polypeptide and that is capable of being translated in the stem cells. Methods for producing RNA molecule capable of translation and methods for introducing such RNA molecules into cells are e.g. known from U.S. Pat. Nos. 6,306,388 and 5,874,268. The intracellular concentration of the exogenously added RNA encoding the Asb-a polypeptide decreases over time by natural turn-over and/or by dilution as a result of cell divisions. Further quantities of exogenous RNA encoding the polypeptide may be added to maintain the intracellular at a level that prevents differentiation until a desired cell population size is reached.

In another embodiment of the method of the invention the exogenous nucleic acid is an expression vector wherein a nucleotide sequence encoding the Asb-a polypeptide is operably linked to a promoter that is capable of regulating transcription in the stem cells. To only temporarily maintain the intracellular concentration of the Asb-a polypeptide at a level that prevents differentiation, the vector preferably is a vector that allows for transient expression of the nucleotide sequence encoding the Asb-a polypeptide. This may be achieved in various ways. For example, the vector may be a vector that is not stably maintained in the stem cells because it lacks an autonomous origin of replication that is functional in the stem cells. Alternatively, the vector may be stably maintained, e.g. by integration into the host cells genome. In that case the transient expression may be achieved by using an inducible promoter or by using a recombination system that allows to remove expression sequences prior to use or differentiation of the expanded cell population.

The expression vector can be any nucleic acid construct comprising a nucleotide sequence encoding Asb-a polypeptide that is suitable for introduction into the stem or progenitor cells, and that is capable of expressing the Asb-a polypeptide upon introduction into these cells. In the expression vector, the nucleotide sequence encoding the Asb-a polypeptide is preferably operably linked to expression signals, such as a signal sequence and transcription regulatory sequence including at least a promoter. The expression signals preferably allow expression of an Asb-a polypeptide encoding nucleotide sequence in the stem or progenitor cells. The promoter is a promoter that is preferably active or can be induced to be active in the stem or progenitor cells. The promoter may be a constitutive promoter, an inducible promoter or a tissue specific promoter, preferably specific for the stem or progenitor cells or a particular lineage of the stem or progenitor cells. Suitable promoters for expression of the nucleotide sequence encoding an Asb-a polypeptide include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter and the human IL-2 promoter. Particularly suitable promoters that are active in stem or progenitor cells include e.g. the Oct4 and Oct5 (Schöler et al., 1989, EMBO J. 8: 2551-2557), TCF-regulated promoters, LIF-regulated promoters, and Notch IC/Her1-targeted promoters. The expression construct may further comprise additional sequence elements for the expression of the nucleotide sequence encoding an Asb-a polypeptide, such as transcriptional enhancers and/or silencers, transcriptional terminators, and polyA-addition sites.

The expression construct may optionally comprise a second or one or more further nucleotide sequence coding for a second or further protein. The second or further protein may be a (selectable) marker protein that allows for the identification, selection and/or screening for Stem or progenitor cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3rd edition), Cold Spring Harbor Laboratory, Cold Spring Harbor Laboratory Press, New York. The nucleotide sequence coding for the marker protein is preferably also operably linked to a promoter for expression in stem or progenitor as described above for the nucleotide sequence encoding an Asb-a polypeptide.

In principle the expression vector may be in the form of any nucleic acid capable of being introduced into stem or progenitor cells. The expression vector may be DNA, RNA or a combination of both; it may be a naked nucleic acid molecule, such as a plasmid or a linear DNA or RNA fragment; and it may be a single or a double stranded nucleic acid molecule. The expression vector may thus be a non-viral vector such as a plasmid or linear nucleic acid that may be packaged in e.g. a liposome for efficient delivery into the stem or progenitor cells. Alternatively, the expression vector is a viral vector that may be used to transduce or infect the stem or progenitor cells. The expression construct preferably is safe, efficient, and reliable and allows for expression, preferably controlled expression of the sequences encoding the Asb-a polypeptide. At present viral vectors are preferred because they are far more efficient agents for gene transfer as compared to the non-viral agents. Suitable viral expression constructs include e.g. vectors that are based on adenovirus, adeno-associated virus (AAV) or retroviruses as recently reviewed by Anderson (1998, Nature 392: 25-30), Walther and Stein (2000, Drugs 60: 249-71) and Kay et al. (2001, Nat. Med. 7: 33-40).

Adenoviral and AAV vectors infect a wide number of dividing and non-dividing cell types. In addition adenoviral vectors are capable of high levels of transgene expression. Moreover, because of the episomal nature of the adenoviral and AAV vectors after cell entry, these viral vectors are particularly suited for application in the present invention wherein only transient expression of the Asb-a polypeptide is required (Russell, 2000, J. Gen. Virol. 2000, 81: 2573-604).

Retrovirus based vectors, on the other hand, may be particularly useful when the target cells are hematopoietic stem or progenitor cells. Retroviral vectors integrate efficiently into the genome of the target cell and they do not transfer any viral gene, which may be an advantage when the expanded cells are implanted in a subject because it alleviates the risk of an immune response against the transgenic cells. A further advantage of the retroviral vectors is their large cloning capacity of up to 8 kb.

Two varieties of retroviral expression constructs may be used in the present invention: oncoretroviral based vectors such as e.g. vectors based on Moloney murine leukemia virus (MMLV) or lentiviral vectors such as e.g. vectors based on human immunodeficiency virus (HIV). A preferred oncoretroviral based expression construct is an MMLV expression construct. MMLV expression constructs are generated from plasmids that contain the gene(s) of interest (i.e. the nucleotide sequence encoding an Asb-a polypeptide and optionally a nucleotide sequence encoding a marker or other second protein, e.g. for regulation of expression of the Asb-a polypeptide) flanked by the two MMLV LTR. Production of the MMLV expression construct requires the use of a packaging cell line to provide the viral proteins necessary for incorporation of the expression construct within viral particle that are capable to infect the stem or progenitor cells. The modified viral particles are replication-defective and retain only one round of infectivity, since they do contain any viral gene. Methods for the construction and use of MMLV expression constructs are described by Anderson (1998, Nature 392: 25-30), and in U.S. Pat. Nos. 5,693,508, 5,817,491, 5,834,256, and 6,017,761.

Alternatively, lentiviral based expression constructs may be applied in the present invention. Lentiviral vectors have the unique ability to infect non-dividing cells (Amado and Chen, 1999, Science, 285: 674-6). This may be an advantage in the present invention because frequently stem cells are isolated from biological sources in a quiescent state. Methods for the construction and use of lentiviral based expression constructs are described in U.S. Pat. Nos. 6,165,782, 6,207,455, 6,218,181, 6,277,633 and 6,323,031 and in Federico (1999, Curr. Opin. Biotechnol. 1999, 10: 448-53) and Vigna and Naldini, (2000, J. Gene Med. 2000 2: 308-16). Retroviral expression constructs may be modified to alter the retroviral vector host range (Marin et al., 1997, Mol Med Today 3: 396-403; Peng and Russell, Curr. Opin. Biotechnol. 10: 454-7).

General recombinant DNA techniques for the construction of the expression vectors and other vectors of the invention are described in Ausubel et al., “Current Protocols in Molecular Biology”, Greene Publishing and Wiley-Interscience, New York (1987) and in Sambrook and Russel (2001, supra); both of which are incorporated herein by reference in their entirety.

The expression vector comprising the nucleotide sequence encoding an Asb-a polypeptide may be introduced into the stem or progenitor cells using a variety of methods available to the skilled person and depending on the nature of the expression construct. Non-viral expression constructs may be used to transform at least part of the stem or progenitor cells using e.g. biolistic transfection, electroporation, or lipofection (see Sambrook and Russel (2001) supra). A convenient means for transfection for use in the present invention is the Nucleofactor™ (www.amaxa.com).

The same methods may be applied to transfect the naked DNAs of viral expression constructs. However, preferably viral expression constructs are packaged into viral particles using the appropriate packaging cell lines and helper viruses and are then used to infect the stem or progenitor cells. Methods for packaging viral expression constructs and subsequent infection of stem or progenitor cells are described in the above listed references on the use of viral expression constructs. Frequently stem cells are isolated from biological sources in a quiescent state. Certain expression vectors, particularly retroviral vectors, do not effectively infect non-cycling cells. Cultures established with these vectors as a source of Asb-a polypeptide sequences are induced to enter the cell cycle by a short period of time in culture with growth factors. For example, hematopoietic stem cells are induced to divide by culture with c-kit ligand, which may be combined with LIF, IL-11 and thrombopoietin. After 24 to 72 hours in culture with cytokines, the medium is changed, and the cells are contacted with the retroviral culture, using culture conditions as described above.

As mentioned above, preferred expression vectors are those that can be removed from the target cells after expansion. This can be accomplished by the use of a transient vector system, or by including a heterologous recombination site that flanks the Asb-a polypeptide coding sequence. In this manner, after expansion the expression construct can be removed prior to use of the expanded cell population. Preferably a detectable marker, e.g. green fluorescent protein, luciferase, cell surface proteins suitable for antibody selection methods, etc. is included in the expression vector, such that after deletion of the construct the cells can be readily isolated that lack the exogenous Asb-a polypeptide.

The term “heterologous recombination site” is meant to encompass any introduced genetic sequence that facilitates site-specific recombination. In general, such sites facilitate recombination by interaction of a specific enzyme with two such sites. Exemplary heterologous recombination sites include, but are not necessarily limited to, lox sequences with recombination mediated by Cre enzyme; frt sequences (Golic et al. (1989) Cell 59:499-509; O'Gorman et al. (1991) Science 251:1351-5; recombination mediated by the FLP recombinase), the recognition sequences for the pSR1 recombinase of Zygosaccharomyces rouxii (Matsuzaki et al. (1990) J. Bacteriol. 172:610-8), and the like. Sequences encoding lox sites are of particular interest for use in the present invention. A lox site is a nucleotide sequence at which the gene product of the cre gene, referred to herein as “Cre,” catalyzes site-specific recombination. A particularly preferred lox site is a loxP site. The sequence of loxP, which is 34 bp in length, is known and can be produced synthetically or can be isolated from bacteriophage P1 by methods known in the art (see, e.g. Hoess et al. (1982) Proc. Natl. Acad. Sci. USA 79:3398). The loxP site is composed of two 13 bp inverted repeats separated by an 8 bp spacer region. Other suitable lox sites include loxB, loxL, and loxR, which can be isolated from E. coli (Hoess et al. (1982) Proc. Natl. Acad. Sci. USA 22:3398).

In an alternative method, expression vectors that provide for the transient expression in mammalian cells may be used. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient short-term expansion of cells, but do not affect the long term genotype of the cell. One way of regulating the maintenance of transient expression vectors is by means of the presence or absence of selective pressure for a selection marker as described above.

In a further embodiment the expression vector may contain a second or further nucleotide sequence (in addition the Asb-a sequence, see above) that encodes a protein that provides for a fail-safe mechanism that allows to cure a the expression vector sequences, in part or in toto, from the cells of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the Asb-a transgenic cells in the subject (see e.g. Clair et al., 1987, Antimicrob. Agents Chemother. 31: 844-849).

In the method of the present invention, a population of cells comprising progenitor and/or stem cells is cultured in vitro in the presence of an Asb-a polypeptide whereby the intracellular concentration of the Asb-a polypeptide is sufficient to prevent differentation of the cells. This intracellular concentration of the Asb-a polypeptide may be achieved either by genetically altering the cells, whereby preferably the genetic alteration is a transient alteration, by providing exogenous Asb-a polypeptide or by providing RNA encoding Asb-a polypeptide, as described above. The upregulation in Asb-a polypeptide is sufficient to maintain or increase the number of assayable stem/progenitor cells in the culture. The number of assayable stem/progenitor cells may be demonstrated by a number of assays. After one week the stem/progenitor cell cloning efficiency will usually be at least about 75% that of the starting cell population, more usually 100% that of the starting cell population, and may be as high as 200% that of the starting cell population.

Following the initial period, there is an increased expansion, where the number of assayable cells having the functional phenotype of the initial cell population can increase from about 5 to about 100 fold or more. After this time, the cells can remain in cycle, and expansion is limited primarily by considerations of space. The cells can be frozen using conventional methods at any time, usually after the first week of culture. Loss of expression will often be sufficient for differentiation to ensue, but may also be actively induced by the appropiate culture protocol and inclusion of one or more differentiation factors (e.g. one of those mentioned above) After seeding the culture medium, the culture medium is maintained under conventional conditions for growth of mammalian cells, generally about 37° C. and 5% CO₂ in 100% humidified atmosphere. Fresh media may be conveniently replaced, in part, by removing a portion of the media and replacing it with fresh media. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

In a another aspect the relates to a stem or progenitor cell comprising a exogenous Asb-a polypeptide and/or an exogenous nucleic acid comprising a nucleotide sequence encoding the Asb-a as described above. The term exogenous is used herein relative to the stem or progenitor cells to indicate that Asb-a polypeptide or nucleic acid do not originate from the stem or progenitor cells. E.g., the Asb-a polypeptide as present intracellularly in the stem cells may have been produced in host cells other than the stem or progenitor cells in which the polypeptide is comprised. The nucleic acid encoding the Asb-a polypeptide may e.g. be a nucleic acid that does not naturally occur in the cells or be in a (genomic) location or genetic configuration that does not naturally occur in the cells. Nevertheless, the sequence of the Asb-a polypeptide or nucleic acid may be identical to the polypeptide or nucleic acid as it naturally occurs in the cells. Alternatively, the term exogenous may indicate a polypeptide or nucleic acid that is heterologous to the stem or progenitor cell. The term “heterologous” when used with respect to a nucleic acid or polypeptide molecule refers to a nucleic acid or polypeptide that one of skill in the art would recognise as heterologous or foreign to the cell in which it is present or expressed. The term heterologous also applies to non-natural combinations of nucleic acid or amino acid sequences, i.e. combinations where at least two of the combined sequences are foreign with respect to each other.

The expanded cells obtained in the methods of the invention may find various applications for a wide variety of purposes. The cell populations may be used for screening various additives for their effect on growth and the mature differentiation of the cells. In this manner, compounds, which are complementary, agonistic, antagonistic or inactive, may be screened, determining the effect of the compound in relationship with one or more of the different cytokines.

The populations may be employed as grafts for transplantation. For example, hematopoietic cells are used to treat malignancies, bone marrow failure states and congenital metabolic, immunologic and hematologic disorders. Marrow samples may be taken from patients with cancer, and enriched populations of hematopoietic stem cells isolated by means of density centrifugation, counterflow centrifugal elutriation, monoclonal antibody labeling and fluorescence activated cell sorting. The stem cells in this cell population are then expanded in vitro and can serve as a graft for autologous marrow transplantation. The graft will be infused after the patient has received curative chemo-radiotherapy. For treatment of neuronal (degenerative) disorders, expanded neuronal precursor cells will be introduced (e.g. by injection in the appropiate neural locus; see e.g. Ramachandran A C, Bartlett L E, Mendez I M. “A multiple target neural transplantation strategy for Parkinson's disease.” Rev Neurosci. 2002; 13(3):243-56.) intracerebrally or in the cerebellum, in the spinal cord or at the appropiate location in the peripheral nervous tissue.

Asb-a polypeptides for use in the present invention can be prepared using recombinant techniques in which a nucleotide sequence encoding the polypeptide of interest is expressed in cultured cells such as described in Ausubel et al., “Current Protocols in Molecular Biology”, Greene Publishing and Wiley-Interscience, New York (1987) and in Sambrook and Russell (2001) “Molecular Cloning: A Laboratory Manual (3rd edition), Cold Spring Harbor Laboratory, Cold Spring Harbor Laboratory Press, New York; both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

Typically, nucleic acids encoding the desired polypeptides are used in expression vectors. Expression vectors decribed above for the expression of Asb-a polypeptides in stem or progenitor cells, as well as expression vectors for recombinant production of Asb-a polypeptides as described below, are both further aspects of the present invention. The phrase “expression vector” generally refers to nucleotide sequences that are capable of affecting expression of a gene in hosts compatible with such sequences. These expression vectors typically include at least suitable promoter sequences and optionally, transcription termination signals. Additional factors necessary or helpful in effecting expression can also be used as described herein. DNA encoding a polypeptide is incorporated into DNA constructs capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines.

DNA constructs prepared for introduction into a particular host typically include a replication system recognised by the host, the intended DNA segment encoding the desired polypeptide and transcriptional and translational initiation and termination regulatory sequences operably linked to the polypeptide encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well-known in the art (see, e.g. Sambrook and Russell, 2001, supra). The transcriptional regulatory sequences typically include a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, e.g., insect cells, e.g. Sf9 cells, mammalian cells, e.g., CHO cells and bacterial cells, e.g., E. coli.

In vitro mutagenesis and expression of mutant proteins are described generally in Ausubel et al. (1987, supra) and in Sambrook and Russell (2001, supra). Also see, Kunkel (1985, supra; describing site directed mutagenesis) and Roberts et al. (1987, supra; describing cassette mutagenesis).

Another method for preparing polypeptides is to employ an in vitro transcription/translation system. DNA encoding a polypeptide is cloned into an expression vector as described supra. The expression vector is then transcribed and translated in vitro. The translation product can be used directly or first purified. Polypeptides resulting from in vitro translation typically do not contain the post-translation modifications present on polypeptides synthesised in vivo. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987 (incorporated herein by reference in its entirety).

In a further aspect the invention thus relates to a host comprising a vector as defined above. The host cells may be prokaryotic or eukarotic host cells as indicated above. The host cell may be a host cell that is suitable for culture in liquid or on solid media. Alternatively, the host cell is a cell that is part of a multicellular organism such as a transgenic plant or animal, preferably a non-human animal. Likewise the host cell may be a stem or progenitor cell as defined above.

A further aspect the invention relates to a method for producing an Asb-a polypeptide. The method comprises the step of culturing a host cell as defined above under conditions conducive to the expression of the polypeptide. Optionally the method may comprise recovery the polypeptide. The polypeptide may e.g. be recovered from the culture medium by standard protein purification techniques, including a variety of chromatography methods known in the art per se.

Another aspect of the invention relates to a transgenic animal comprising in its somatic and germ cells a vector as defined above. The transgenic animal preferably is a non-human animal. Methods for generating transgenic animals are e.g. described in WO 01/57079 and in the references cited therein. Such transgenic animals may be used in a method for producing a Asb-a polypeptide, the method comprising the step of recovering a body fluid from a transgenic animal comprising the vector or a female descendant thereof, wherein the body fluid contains the polypeptide, and, optionally recovery of the polypeptide from the body fluid. Such methods are also described in WO 01/57079 and in the references cited therein. The body fluid containing the polypeptide preferably is blood or more preferably milk.

Yet another aspect of the invention relates to a transgenic plant comprising in its cells a vector as defined above. Methods for generating transgenic plants are e.g. described in U.S. Pat. No. 6,359,196 and in the references cited therein. Such transgenic plants may be used in a method for producing an Asb-a polypeptide, the method comprising the step of recovering a part of a transgenic plant comprising in its cells the vector or a part of a descendant of such transgenic plant, whereby the plant part contains the polypeptide, and, optionally recovery of the polypeptide from the plant part. Such methods are also described in U.S. Pat. No. 6,359,196 and in the references cited therein.

Another aspect of the invention relates to an antibody or antibody-fragment that specifically binds to an Asb-a polypeptide as defined above. Methods for generating antibodies or antibody-fragments that specifically binds to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

The aspects of the invention are illustrated by means of the following Examples.

### DESCRIPTION OF THE FIGURES

FIG. 1: Asb-a binding to Notch signalling proteins. Co-transfections in HEK-293 cells were followed by co-immunoprecipitations and immunoblotting. Immunoblots are shown with controls to monitor expression of the proteins, as indicated. (a) Overview of the constructs used. (b) Homodimerization of Asb-a Full length MT-Asb-a and MT-Asb-aΔC co-immunoprecipitated with HA-Asb-a, HA-Asb-aΔC and HA-Asb-aΔSOCS. (c) MT-Notch1a IC co-immunoprecipitated with HA-Asb-a, HA-Asb-aΔC and HA-Asb-aΔSOCS. (d) Mammalian flag-tagged RBP3 (Flag-RBP) did not co-immunoprecipitate with HA-Asb-a. (e) MT-Nrarp co-immunoprecipitated with HA-Asb-a. WCL, whole cell lysate.

FIG. 2: Asb-a inhibits the NGF-induced block in cell proliferation until at least 3 days. (A) Relative mitochondrial activity was measured (MTT assay) for control (MT), MT-Notch IC and MT-Asb-a transfected PC12 cells. The experiments were performed in duplicate and the values shown, are averages and SEM of 11 different samples (**: p<0.01). (B) PCNA protein expression levels are lowered in MT and MT-Notch IC transfected PC12 cells, upon 2 days of NGF stimulation, but not in the cells overexpressing MT-Asb-a. PCNA expression was detected by SDS-PAGE and immunoblotting with the anti-PCNA antibody.

FIG. 3: Notch IC and Asb-a overexpression inhibit the formation of NGF-induced neurite extensions. Neurite length was measured of at least 10 different fields of cells for each condition. The average neurite length per somata of PC12 cells that were stimulated with NGF was reduced in cells transfected with MT-Notch or MT-Asb-a as compared to the control as assessed by computational measurement of neurite length (see materials and methods 2.1). The graph therefore clearly shows that Notch and Asb-a both strongly inhibit neurite formation (**: p<0.01).

FIG. 4: Asb-a overexpression maintains cells in a neuronal precursor stage. The protein expression profile of GAP43 and neurofilament of whole cell lysates of PC12 cells transfected and selected on neomycin with MT (control), MT-Notch IC or MT-Asb-a upon stimulation with 100 ng/ml NGF was analysed. PC12 cells were stimulated with NGF and after 0, 24 and 48 hours cells were lysed and analysed by SDS-PAGE and immunoblotting. The different proteins were detected on parallel blots. Equal amounts of protein per lane were confirmed by visualizing the expression levels of β-actin.

FIG. 5: Transcriptional activity of different transcription factors upon stimulation is decreased upon Asb-a overexpression. HeLa cells were co-transfected with MT-Asb-a and a reporter construct for various transcription factors. After the transfection, cells were stimulated with the relevant stimulus and secreted alkaline phosphatase (SEAP) amounts in the medium were measured after 0 and 24 hours. Asb-a markedly down regulates transactivation of Interferon (A), cAMP (B), c-fos/c-jun (C) and NF-κB (D) reporter elements. The data shown is an average and SEM of at least three different experiments and every condition was performed in quadruplo (**: p<0.01).

FIG. 6: Asb-a overexpression results in a SOCS box-dependent ubiquitination of IκBα(A) and p65 (B) immunoprecipitates. HeLa cells were transfected with either MT alone, MT-Notch IC, MT-Asb-a or MT-Asb-aΔC in combination with HA-ubiquitin. Prior to stimulation, the proteasome inhibitor MG-132 was added (10 μM) and cells were stimulated for 60 minutes with 10 ng/ml Il-1β. Equal amounts of protein were somata immunoprecipitated with anti-IκBα(A) or p65 (B) antibody and subsequently analysed for the presence of ubiquitinated proteins by immunoblotting with ubiquitin antibody.

FIG. 7: Asb-a interacts with IκBa in vitro. HEK-293 cells were transfected with HA-Asb-a and/or IκBα(middle and lower panel). Equal amounts of cell lysates were immunoprecipitated with the anti-HA antibody and cells were analysed for IκBα expression. IκBα was detected in the precipitate of HA-tagged proteins (upper panel).

### EXAMPLES

### 1

### Example 1

### Isolation and Characterisation of the Zebrafish Asb-a Gene

### 1.1 Method and Materials

1.1.1 Fish and Embryos

Zebrafish were kept at 27.5° C. Embryos were obtained by natural matings, cultured in embryo medium (Westerfield, 1994) and staged according to Kimmel et al. (1995).

1.1.2 In Situ Hybridisation

Whole mount in situ hybridisations were performed according to Joore et al. (1994). For histological analysis, embryos were fixed for 1 hour in 0.2% glutaraldehyde, 4% paraformaldehyde in phosphate buffer, dehydrated, embedded in plastic and sectioned.

1.1.3 RNA Isolation and Northern Blot Analysis

Total RNA was extracted using the single-step method for RNA isolation by acid guanidinium thiocyanate-phenol-chloroform as described by Chomczynski and Sacchi (1987). Total RNA (20 μg) was used for Northern blot analysis, that was performed using standard techniques.

1.1.4 cDNA Library Screen

A random primed λZAP neurula cDNA library prepared from 3 to 15 hpf embryos was used to screen 1.5×10⁶ phages to obtain a full-length clone. The probe was labelled to high specific activity with α-[³²P]-dCTP (Amersham) using a rediprime labelling kit (Amersham). After in vivo excision, the longest positive clone was subcloned and sequenced.

1.1.5 Plasmid Construction

To obtain an expression vector containing asb-a, the coding region was cloned into the BamHI/XhoI sites of the pCS2⁺ expression vector. To obtain a myc-tagged version of asb-a (MT-asb-a), the coding region cloned into the NcoI/XhoI sites of pCS2⁺MT. A MT-asb-aΔC construct encoding a C-terminal deletion (aa 229-293) was generated by digesting MT-asb-a with XbaI. For HA-tagged asb-a (HA-asb-a), the coding region was cloned into the XhoI/EcoRI sites of pMT2SM-HA. For the glutathione S-transferase (GST) fusion construct, the coding region was cloned into the BamHI/EcoRI sites of pGEX-KG. To obtain a construct for RNA synthesis of asb-a, the 1.6 kb asb-a cDNA was cloned into the BglI and XhoI sites of pT7TS⁺. A partial cDNA fragment of asb-a in pBluescript was used as a template to generate a riboprobe for in situ hybridisations. For myc-tagged Nrarp, zebrafish nrarp (AI 957982) was cloned into the EcoRI and XbaI sites of pCS2⁺MT.

1.1.6 RNA Synthesis and Microinjection

Capped mRNAs were synthesised using the mMESSAGE mMACHINE kit (Ambion). Approximately 300 pg mRNA (1 nl) was injected.

1.1.7 Rabbit Immunisation and Serum Preparation

GST-tagged Asb-a was purified from E. coli BL21 bacteria using glutathione sepharose beads. Rabbits were immunised with GST-tagged Asb-a. Subsequently, the serum containing the polyclonal antibody directed against Asb-a was used in immunoblotting analysis.

1.1.8 Zebrafish Cell Lysate for Immunoblotting

Embryos were lysed in cell lysis buffer (50 mM HEPES pH7.5, 150 mM NaCl, 1.5 mM MgCl₂, 1 mM EGTA, 10% glycerol,1% Triton X-100) at 4° C. (7.5-12.5 μl/embryo). Loading buffer was added to the cell lysis buffer and samples were boiled for 3 min. Per lane, a total equivalent of 5 embryos was loaded.

1.1.9 Cells, Transfections, Immunoblotting and Immunofluorescence

COS-7 and HEK-293 were cultured in Dulbecco's Modified Eagle's Medium (DMEM) supplemented with 7.5% bovine calf serum. Cells were transfected using calcium-phosphate precipitation. After transfection, COS cells were lysed in cell lysis buffer and loaded on SDS-polyacrylamide gels. The material was transferred to Immobilon (Millipore) by semi-dry blotting in transferbuffer (50 mM Tris pH 8.0, 40 mM Glycine, 0.0375% SDS, 20% methanol) and the blots were incubated in blocking buffer (50 mM Tris pH 8.0, 150 mM NaCl, 0.05% Tween-20, 5% non-fat milk) overnight at 4° C. Blots were incubated in TBS-T (50mM Tris pH 8.0, 150 mM NaCl, 0.05% Tween-20) containing 10% blocking buffer and rabbit polyclonal anti-asb-a serum or mouse anti-MT antibody (9E10, Santa Cruz Biotechnology) for 1 h and washed in TBS-T. After incubation with the appropriate secondary antibody and extensive washing in TBS-T, the blots were developed by ECL.

For subcellular localisation, transiently transfected COS-7 cells were fixed in 2% paraformaldehyde and permeabilised with 0.1% Triton X-100. After blocking with 0.5% BSA, cells were incubated with anti-MT antibody (9E10) in 0.5% BSA. After extensive washing the cells were incubated with TRITC-conjugated secondary anti-mouse antibody. Finally, the cells were washed and the fluorescence was examined with a confocal laser scanning microscope.

For co-immunoprecipitations, transiently transfected HEK-293 cells were used. Immunoprecipitation of protein complexes using anti-HA-epitope antibody 12CA5 was performed using standard techniques.

1.1.10 Whole Mount Immunofluorescence and Immunohistochemistry

Fixed embryos were washed in PBST (PBS containing 1% Tween-20) and blocked overnight in block buffer (PBST containing 10% heat inactivated sheep serum and 0.5% blocking reagent (Boehringer)) at 4° C. Block buffer was discarded and anti-MT (9E10) antibody was added (1:500) and incubated overnight at 4° C. After washing (10 times 10 min in PBST), the embryos were incubated in the secondary antibody, anti-mouse-Cy3 (1:250 in block buffer), overnight at 4° C. Fluorescence was analyzed on a confocal microscope.

For whole mount immunohistochemistry, fixed embryos were rinsed in PBS and incubated in blocking solution for 1 hour (PBST containing 1% BSA and 2% normal lamb serum). The primary antibody (anti-Hu, Sigma) was added and embryos were incubated overnight at 4° C. After washing in PBST, embryos were incubated overnight at 4° C., in goat anti-mouse peroxidase-conjugated secondary antibody. After extensive washing, embryos were incubated in 0.5 mg/ml DAB and then reacted in 0.003% H₂O₂.

1.1.11 Acridine Orange Staining

Acridine orange staining was performed as described (Furutani-Seiki et al. 1996) with modification of the acridine orange (Sigma) solution; 0.078 μg/ml in embryo medium.

1.1.12 Tunel Assay

Tunel assays were performed as described by Cole and Ross (2001) with modification of the staining reaction. Anti-DIG antibody was preabsorbed in blocking buffer (1% DMSO and 2% BSA in PBS) for 1 hour at 4° C. Embryos were incubated overnight in blocking buffer with 1:2000 anti-DIG antibody at 4° C., washed 8 times with PBS and stained according to the method used for in situ hybridisation.

1.1.13 Morpholinos Antisense morpholinos were obtained from Gene Tools, LLC. The sequence was as follows: Asb-a-MO, 5′-AGAACCTCGCAGACAGCAACGGTC-3′. Approximately 4 to 5 ng was injected into one-cell stage embryos.

1.1.14 3D-Reconstruction and Calculation

Volumes were calculated from a 3D reconstruction from 7 μm serial sections with an acquisition station (Verbeek and Boon, 2002). This resulted in 123 section images for the control and 149 section images for the treated fish. The notochord and the otic vesicle were included to have a frame of reference.

### 1.2 Results

1.2.1 Isolation and Characterisation of the Zebrafish Asb-a Gene

We recovered a cDNA fragment from a differential display screen designed to isolate genes that are involved in zebrafish neural plate regionalisation. A full-length cDNA was obtained and sequenced. The largest ORF encodes 293 amino acids (Table 2). The amino acid sequence contains six ankyrin repeats (Tabel 3; Lux et al., 1990), motifs that mediate protein-protein interactions (Bork, 1993; Sedgwick and Smerdon, 1999) and a conserved carboxy-terminal motif termed suppressor of cytokine signalling (SOCS) box that was initially identified in the SOCS family of proteins (Table 4; Hilton et al., 1998). Alignment of the amino acid sequence identified members of the family of ASB proteins that contain a variable number of ankyrin repeats and a C-terminal SOCS box (Kile et al., 2000) with highest homology to human and murine ASB-5, -9 and -11 ranging between 40.7 and 43.7 percent identity (Table 2). Since we could not assign the zebrafish gene to any of the vertebrate homologs based on the sequence (Table 2) and phylogenetic tree (Table 5), we named the identified gene asb-a.

1.2.2 Expression Pattern of asb-a mRNA and Asb-a Protein

Expression of asb-a was first detected at 4 hpf (hour post fertilisation) and was ubiquitous throughout the blastoderm (data not shown). Asb-a was not maternally expressed since the transcripts were not detected by Northern blotting in 2.5 hpf embryos (data not shown). During late gastrula, the ubiquitous expression disappeared and asb-a transcripts were localised to the polster (data not shown). At the end of gastrulation the expression was confined to the polster and the lateral margins of the neural plate where primary sensory neurons are born and subsequently the neural crest originates. Moreover, two anterior stripes extended from the margins towards the midline at the level of prospective mid/hindbrain boundary (data not shown). Weaker expression was observed in paraxial bilateral domains that likely corresponded to the PSM (data not shown). The axial midline region was devoid of asb-a transcripts (data not shown). Sections through the margin of the neural plate demonstrated that in this region cells from all three germ layers contained asb-a transcripts (data not shown). In contrast, in the bilateral paraxial domains, asb-a transcripts were restricted to cells of the hypoblast only (data not shown). At the onset of somitogenesis the level of transcripts increased and extended throughout paraxial regions. The expression was excluded from the midline as well as from stripes in paraxial domains that form a segmented pattern (data not shown). The characteristic pattern persisted for several hours to become undetectable from 12 somites to at least 3 dpf (divisions post fertilisation), although low levels of mRNA were still detected in 24 hpf embryos by Northern blotting (data not shown). In conclusion, asb-a localised to specific domains of the embryo being reminiscent of expression of Delta-Notch genes in fish and frog neurula embryos (Bierkamp and Campos-Ortega, 1993; Domseifer et al., 1997; Westin and Lardelli, 1997; Haddon et al., 1998b; Kortschak et al., 2001; Lamar et al., 2001). For instance, a comparison with the expression pattern of notchla revealed the existence of overlapping as well as mutually exclusive domains between asb-a and notchla (data not shown). Moreover, in the polster notchla and asb-a transcripts were shared by the same population of cells (data not shown).

To analyse protein expression of Asb-a, an anti-Asb-a polyclonal antibody was raised against a full length GST-tagged Asb-a fusion protein. COS cells transfected with MT-Asb-a as well as zebrafish embryos injected with MT-Asb-a RNA (800 pg), allowed detection of MT-Asb-a by immunoblotting with the anti-Asb-a antibody (45 kDa). As a control, the blot was reprobed with the MT antibody (9E10) and proteins of the same size were detected which confirmed that the anti-Asb-a antibody specifically recognised Asb-a (data not shown). In COS cells transfected with an expression vector for Asb-a, Asb-a was expressed as a protein of approximately 32 kDa (data not shown).

Since the Asb-a amino acid sequence contains ankyrin repeats which have been documented to be present in several transcriptional (co)-regulators such as cactus/IkB or notch IC (Bork, 1993) we investigated the subcellular localisation of MT-Asb-a. Upon transfection into COS cells, MT-Asb-a was found predominantly in the nucleus while a very weak signal was observed throughout the cytoplasm (data not shown). Control experiments showed that MT alone was not localised in COS cells (data not shown). Nuclear localisation of Asb-a is compatible with a possible role in transcriptional regulation.

1.2.3 Homodimerisation of Asb-a and Interaction with Notch1a IC and Nrarp

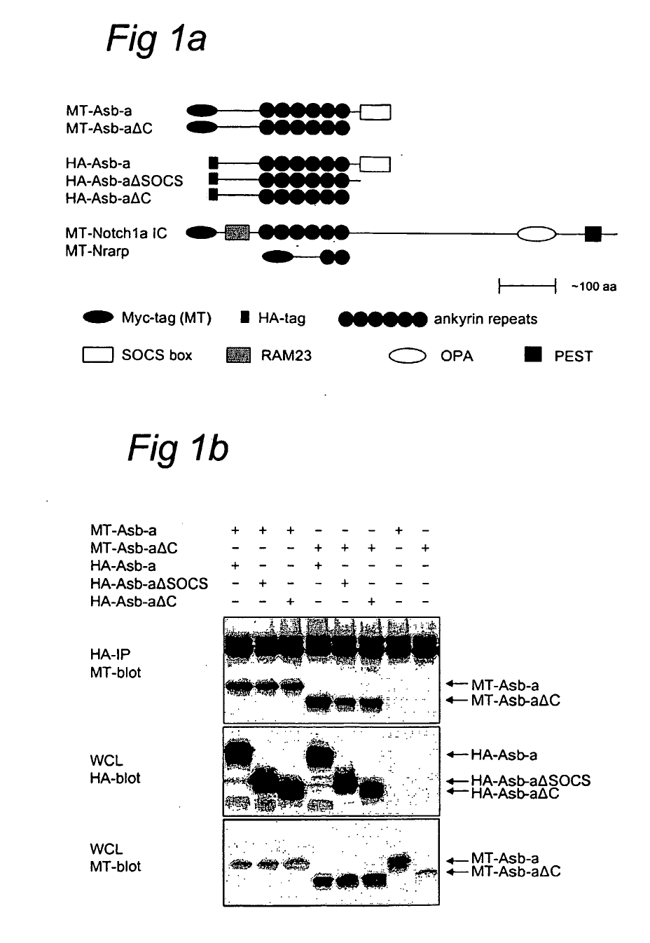

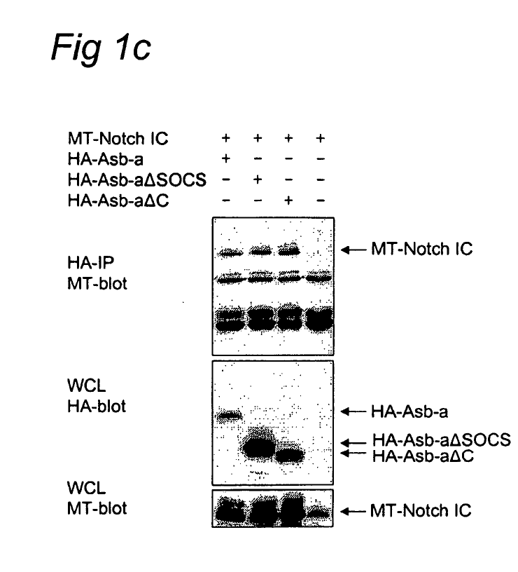

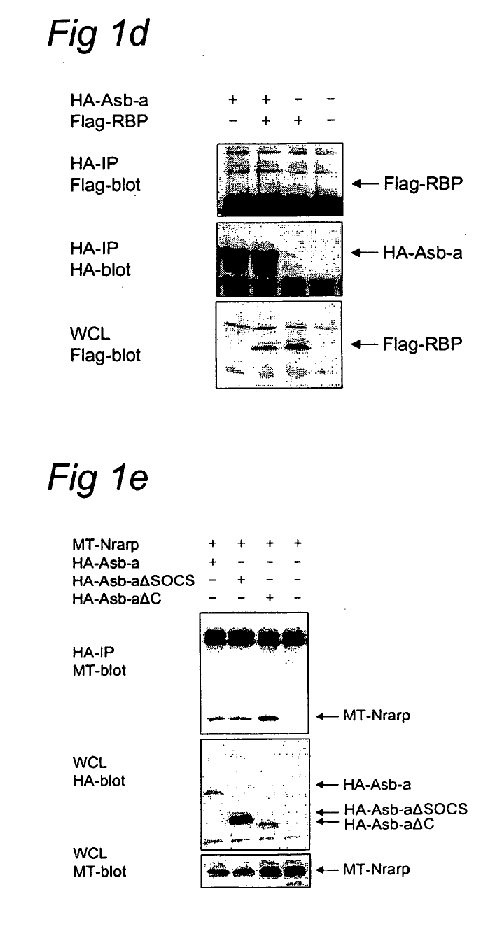

To study whether Asb-a has dimerisation properties similar to other ankyrin repeat-containing proteins (Sedgwick and Smerdon, 1999; Matsuno et al., 1997), we co-transfected MT-Asb-a and HA-tagged Asb-a (HA-Asb-a) (FIG. 1A). Upon immunoprecipitation of HA-tagged proteins we identified MT-Asb-a by immunoblotting, demonstrating the homodimerisation capacity of the Asb-a protein (FIG. 1B). We investigated interaction capacities of truncated forms of Asb-a to establish the site of interaction. Asb-aΔSOCS and Asb-aΔC encode Asb-a with deletions of the SOCS box or of the entire carboxy-terminal part (FIG. 1A). Co-immunoprecipitation experiments demonstrated that full length Asb-a as well as Asb-aΔC and Asb-aΔSOCS interacted with full length Asb-a, Asb-aΔC and Asb-aΔSOCS (FIG. 1B), indicating that homodimerisation of Asb-a was not dependent on the SOCS box, but was mediated by the ankyrin repeats.

Since Notch IC contains six ankyrin repeats, like Asb-a, we investigated whether Asb-a bound zebrafish Notch1a IC. Indeed, we detected co-immunoprecipitation of myc-tagged Notch1a IC with HA-Asb-a (FIGS. 1A and 1C).

Because of the association between Asb-a and Notch IC, we decided to investigate the interaction of Asb-a with other proteins that are involved in Delta-Notch signalling. Notch target genes are regulated upon association of Notch IC with CSL (CBFI, Suppressor of hairless (Su(H), Lag-1; Mumm and Kopan, 2000). We tested whether Asb-a could bind RBP, the mammalian homologue of Su(H). Although transfected Flag-tagged RBP3 protein was present in whole cell lysates, we did not detect the Flag-tagged RBP3 protein in Ha-Asb-a immunoprecipitates (FIG. 1D). Asb-a does not contain a RAM domain like Notch IC. The Notch IC RAM domain is necessary for binding to CSL and therefore the finding that Asb-a did not bind RBP3 is not surprising. These results indicate that Asb-a may function in the Notch pathway, without binding directly to CSL.

Another component of the Notch signalling pathway is Nrarp, which contains two ankyrin repeats (Krebs et al., 2001; Lamar et al., 2001; Lahaye et al., 2002). Since proteins containing ankyrin repeats bind other proteins containing ankyrin repeats, we tested whether Nrarp bound Asb-a. An EST containing the putative zebrafish Nrarp sequence was identified. Myc-tagged Nrarp indeed co-immunoprecipitated with Asb-a (FIGS. 1A and 1E). Asb-aΔC and Asb-aΔSOCS associated with MT-Nrarp as well, indicating that the ankyrin repeats were responsible for the interaction (FIG. 1E).

The fact that asb-a and notchla IC are expressed in overlapping domains and that Asb-a bound directly to Notch1a IC as well as Nrarp suggests that functional interactions of these proteins are likely to occur within multiprotein complexes in the developing embryo.

1.2.4 Asb-a Overexpression Affected her1 Expression, Somite Formation and Primary Neurogenesis

Since Asb-a bound Notch1a IC, we investigated whether asb-a interfered with downstream events of Notch-Delta signalling. We assessed expression of her1, a target of Notch belonging to the Hairy and Enhancer of split genes (HES/E(spl)), upon microinjection of MT-asb-a mRNA (data not shown) into one of the blastomeres of 2-cell stage embryos. Her1 is expressed in the anlagen of consecutive somites in the PSM (data not shown; Holley et al., 2000). Following MT-asb-a overexpression in one half of the embryo, her1 expression was affected as shown by 4 categories (Table 1). The highest percentage phenotype showed thinner segmental stripes of her1 transcripts in the somites (35.5%, n=62) (data not shown) indicating that overexpression of asb-a interfered with segmental cyclical expression of her1 during somitogenesis. Diffuse expression or expression driven towards the midline was reminiscent of the effect of Notch IC overexpression on her1 in zebrafish embryos (Takke and Campos-Ortega, 1999), and was observed in lower percentages (Table 1). The affected her1 pattern coincided with the distribution of the MT-Asb-a protein in one half of the embryo (data not shown).

To study to what extent the effect of Asb-a overexpression on her1 expression was mediated by the SOCS box present in Asb-a, we overexpressed the deletion construct that lacked the C terminal part of the protein including the SOCS box (data not shown). Overexpression of MT-asb-aΔC mRNA resulted in similar phenotypes as obtained by overexpression of full length MT-asb-a, albeit in different frequencies (Table 1). These data show that asb-a overexpression influenced expression of the Notch target gene her1 and that the SOCS box may contribute to regulation of the Notch signal.

Since asb-a overexpression interfered with her1, we investigated whether it would affect somitogenesis as Notch signalling is required for the establishment of somites and their boundaries (Bierkamp and Campos-Ortega, 1999; van Eeden et al., 1996; Jiang et al., 2000; Pourquie, 2000; Holley et al., 2002). The commitment of cells to the myogenic lineage is evident from their expression of myoD in adaxial cells and in the posterior region of each developing somite (data not shown; Weinberg et al., 1996). Overexpression of asb-a mRNA resulted in the loss of somitic myoD expression accompanied by a variably reduced or absent expression in adaxial cells (Table 1) or in a bended and/or displaced field of myoD expression (data not shown). When MT-asb-aΔC was overexpressed, we observed similar classes of defects in the myoD pattern with no significant difference in their frequency (Table 1). Interestingly, the expression pattern of MT-Asb-a in the embryo coincided with exclusion of myoD expression (data not shown), suggesting that Asb-a may function to prevent the myogenic differentiation program.

In addition to its involvement in somitogenesis, Notch/Delta signalling has been implicated in lateral inhibition during primary neurogenesis (Appel and Eisen, 1998; Baker, 2000). Ectopic activation of Notch signalling in zebrafish embryos leads to reduced numbers of primary neurons as shown by islet-1 labelling (Dornseifer et al., 1997; Takke and Campos-Ortega, 1999). In zebrafish, islet-1 labels the polster, the cranial ganglia, Rohon-Beard neurons and primary motorneurons (data not shown; Korzh et al., 1993; Inoue et al., 1994) and these domains overlap with the pattern of expression of asb-a (data not shown).

We investigated whether overexpression of Asb-a would interfere with primary neurogenesis, by overexpression of MT-asb-a MRNA and evaluation of islet-1 (Table 1). Indeed in 80% of the embryos (n=66) islet-1 expression was affected (data not shown). Most prominent was a reduction or total absence of islet-1 expression in the lateral sensory Rohon-Beard neurons (data not shown) being reminiscent of Notch activation (Dornseifer et al., 1997; Takke and Campos-Ortega., 1999). Occasionally, defects in the ventromedial motorneurons or the cranial ganglia were observed (data not shown). Overexpression of MT-asb-aΔC also resulted in reduction of the number of neurons, although in a significantly lower percentage (58.5%, n=44). Immunolabelling demonstrated that MT-Asb-a was present in the domains that did not contain islet-1 labelling (data not shown). The results indicate that the SOCS box of Asb-a may contribute to disruption of primary neurogenesis.

Together the data indicate that in overexpression experiments Asb-a interfered with Notch signalling as assessed by her1, myoD and islet-1 expression. In processes such as primary neurogenesis and somitogenesis Asb-a appears to mimick overexpression of notch1a IC. Importantly, Asb-a appears to block differentiation as displacement of the myoD and islet-1 expression was found outside the MT-Asb-a domain. Although we see that deletion of the SOCS box changes the frequency of molecular phenotypes with respect to the her1 and islet-1 expression patterns, its precise role could not be unambiguously determined in overexpression experiments.

1.2.5 Morpholino-Mediated Asb-a Knockdown Induced a Relative Increase of Neurons in the Neural Tube

Since asb-a is expressed in regions of the embryo that correspond to neurogenic domains and since overexpression of full length asb-a MRNA abolished primary neurons, we reasoned that the function of Asb-a might be similar to that of neurogenic genes in Drosophila whose loss of function leads to supernumerary neurons (Knust and Campos-Ortega, 1989). We tested this by generating knockdown embryos by injection of morpholinos (Nasevicius and Ekker, 2000) designed to inactivate Asb-a (Asb-a-MO). To establish whether Asb-a-MO abolished Asb-a protein we carried out immunoblotting of proteins isolated from Asb-a-MO injected and non-injected embryos using the polyclonal antibody against Asb-a. Immunostaining of proteins from 12 hpf wildtype embryos detected transient expression of the 29 kDa Asb-a protein whereas the Asb-a protein was absent in morphants (data not shown) demonstrating the morpholino-mediated block of translation of asb-a mRNA. At 3 dpf, morphants were typically smaller with a shortened trunk, sometimes accompanied by a downward directed curly tail and a hyperpericardium (data not shown). When tested for their touch response (Granato et al. 1996), Asb-a morphants appeared less sensitive (80%, n=85) as morphants responded by a jerky upright movement and immediately settled down at the spot again whereas wildtype embryos swam away.

To investigate whether neurogenesis was affected in Asb-a morphants, we assessed expression of the proneural bHLH gene neurogenin1 (ngn1) that is negatively regulated by Notch activity (Blader et al., 1997; Korzh et al., 1998; Kim et al., 1997; Appel et al., 2001; Cornell and Eisen, 2002). Cells expressing ngn1 reside in the brain and in the neural tube where neurons are born (Appel and Eisen, 1998; Korzh et al., 1998). In the neural tube of morphants, ngn1 was expressed at higher levels. In addition, ngn1 expression domains appeared expanded (data not shown). The data suggest that the targeted knockdown of Asb-a interfered with neuronal differentiation.

To study whether the observed increase in ngn1 mRNA upon knockdown of Asb-a resulted in induction of supernumerary neurons, we analyzed the expression of HuC, a marker for postmitotic neurons, at 24 hpf, 48 hpf and 72 hpf. At 24 hpf, a proportion of Hu positive cells in the neural tube appeared higher in morphants as compared to wildtype embryos (data not shown). The difference between postmitotic neurons and unlabelled cells was obvious at 48 hpf with a relatively low number of unlabelled cells in the neural tube of the morphants (data not shown). At 72 hpf the difference became striking with almost all cells in the neural tube being Hu-positive (data not shown).

To establish the proportion of post-mitotic neurons relative to presumably proliferating unlabelled precursors that are in the ventricular zone of the CNS, a 3D reconstruction of serial sections from a 24 hpf wildtype embryo and a representative 24 hpf morphant was made. The measurements show that Hu-positive cells contribute 28 Vol % of the neural tube in the wildtype embryo and 36 Vol % in the morphant (data not shown). When these relative volumes are normalised to the wildtype volume we find: 34 Vol % of post-mitotic neurons in the morphant as compared to 28 Vol % in the wildtype embryo. Since cell differentiation and programmed cell death are tightly coupled events and Notch signalling has been implicated in cell fate decisions including entry into apoptosis (Miele and Osborne, 1999), we analyzed whether the increase of Hu-negative cells in the morphants was accompanied by apoptosis in the CNS. We found enhanced apoptosis in 24 hpf morphants using acridine orange (AO) as an indicator of apoptosis (Furutani-Seiki et al., 1996). In wildtype embryos very few apoptotic cells were present (data not shown) whereas in the morphants an increase in their number in the brain and neural tube occured (data not shown). Analysis of apoptotic cells was confirmed using TUNEL assay (data not shown). Transversal sections of the neural tube showed random apoptotic cells in the neural tube of morphants (data not shown) indicating that the cell death was an early event initiated prior to terminal differentiation.

### 2 Example 2

### Overexpression of the Zebrafish Asb-a Gene Inhibits Differentiation

### 2.1 Methods and Materials

2.1.1 Cell Cultures

HeLa and PC12 cells were maintained in Dulbecco's Modified Eagle's Medium (DMEM) containing 10 and 15% FCS respectively. The culture medium was supplemented with 5 mM glutamine and antibiotics/antimitotics. Cells were incubated in a 5% CO₂ humidified incubator at 37° C. Human embryonic kidney cells (HEK-293) were cultured in DMEM supplemented with 7.5% FCS in a 7.5% CO₂ incubator.

2.1.2 Expression Constructs

pCS2+MT (MT; myc tagged expression vector), pCS2+MT-Asb-a (MT-Asb-a), pCS2+MT-Asb-aΔC (MT-Asb-aΔC), pMT2SM-HA-Asb-a (HA-tagged expression vector; HA-Asb-a) as described above in Example 1, HA-ubiquitin (gift of G. Strous), pCS2+MT-Notch1a IC (MT-Notch IC; gift of J. A. Campos-Ortega), the IκB-α expression construct was purchased from Clontech (PT3429-5).

2.1.3 Immunofluorescence

PC12 cells were transfected with MT alone, MT-Notch IC or MT-Asb-a plus a neomycin resistance plasmid. After 5 days, the polyclonal neomycin-resistant PC12 cells were reseeded on glass coverslips and stimulated with 100 ng/ml nerve growth factor (NGF-2.5S) (Invitrogen). The cells were fixed in 4% paraformaldehyde for 15 min and kept in PBS at 4° C. After fixation, the cells were treated with PBS+0.1% Triton X-100 for 5 min and subsequently blocked with PBS containing 5% Normal Goat Serum. After blocking, the cells were incubated with 1:100 MT antibody (Cell Signal #2272) in PBS+3% BSA and afterwards incubated with 1:5000 anti-Rabbit-Cy3. Following the first staining, the cells were stained for neurofilament identical to the MT-staining but with anti-mouse-Cy5 as secondary antibody. The cells were washed three times in PBS between every step. After staining the glass coverslips were mounted in moviol.

2.1.4 MTT Assay

PC12 cells were transfected with MT alone, MT-Notch IC or MT-Asb-a plus neomycin resistance plasmid. After 5 days, polyclonal neomycin-resistant PC12 cells were reseeded in 96 well plates and stimulated with 100 ng/ml nerve growth factor. After three or six days 0.1 mg MTT (Sigma M5655) was added to the medium. The cells were lysed by addition of 50 μl MMT lysis buffer (20% SDS/10% DMF) to the medium. After complete lysing of the cells, the absorbance was measured at 570 nm. The results are the mean and standard error of 11 independent cultures obtained in 2 different experiments.

2.1.5 Measurement of the Neurite Length

The average neurite length was measured by blinded counting of the total amount of neurites divided by the amount of cells. For blindly counting of the neurites, we used the Image Analysis program from EFM Software (Rotterdam, The Netherlands).

2.1.6 Immunoblotting

Cells were seeded in 6-well plates to be at 50% confluency at the time of stimulation. After the stimulation the cells were washed in ice cold PBS and taken up in 100 μl sample buffer and heated to 95° C. for 5 minutes. Cell lysates were separated on a SDS-PAGE gel and blotted onto a PVDF membrane. Antibody probing was performed according to the supplier's protocol.

2.1.7 Transactivation of Reporter Constructs

PC12 and HeLa cells were seeded in 96-wells plates at 20% confluency and transfected using effectene (Qiagen), according to the supplier's protocol, with MT alone, MT-Asb-a or MT-Asb-aΔC plus the different transcription factor responsive constructs (ISRE, CRE, AP1 and NF-κB) coupled to a secreted alkaline phosphatase (SEAP) as the reporter protein. The following day the medium was changed for growth medium containing 2.5% serum. After 24 h the cells were stimulated with the positive control for the specific transcription factor. After 0 and 24 hours a sample of the medium was assayed for the presence of SEAP as described previously (Bronstein et al, 1994).

In short, after collection the SEAP samples were spun down and the supernatant was subsequently incubated at 65° C. for 30 minutes. The sample was then placed on ice and 25 μl of the sample was mixed with 25 μl PBS and 50 μl of 2×SEAP buffer (2 M diethanolamine pH 9.8, 1 mM MgCl₂, 20 mM L-homoarginine). The addition of PBS lowered the fluorescence of the sample but also increased the linear range of detection. The samples were incubated at 37° C. for 10 minutes. Subsequently 50 μl 1×SEAP buffer, containing 120 μM 4-methylumbelliferylphosphate (MUP), was added to the mixture and fluorescence was measured at 360/460 nm after different time points using a cytofluormeter (Perseptive systems).

2.1.8 Transfection and Immunoprecipitation in HEK-293 Cells

Cells were transfected using calcium-phosphate precipitation. After transfection, HEK-293 cells were lysed in cell lysis buffer. Immunoprecipitation of protein complexes using anti-HA-epitope antibody 12CA5 was performed using standard techniques. Whole cell lysate and immunoprecipition samples were loaded on SDS-polyacrylamide gels. The material was transferred to Immobilon (Millipore) by semi-dry blotting in transferbuffer (50 mM Tris pH 8.0, 40 mM Glycine, 0.0375% SDS, 20% methanol) and the blots were incubated in blocking buffer (50 mM Tris pH 8.0, 150 mM NaCI, 0.05% Tween-20, 5% non-fat milk) overnight at 4° C. Blots were incubated in TBS-T (50 mM Tris pH 8.0, 150 mM NaCl, 0.05% Tween-20) containing 10% blocking buffer and the IκBα-antibody (1:1000). After incubation with the secondary antibody and extensive washing in TBS-T, the blots were developed by enhanced chemiluminescence (ECL).

2.1.9 Ubiquitination Assay in HeLa Cells

For the determination of the presence of ubiquitinating activity of Asb-a, HeLa cells were transfected using effectene with the MT, MT-Notch IC, MT-Asb-a or MT-Asb-aΔC. Additionally, an expression construct containing HA-ubiquitin was transfected. Three hours prior to IL-1β stimulation, the cells were treated with 10 μM MG-132. After 60 minutes stimulation, the cells were lysed in cell lysis buffer (Cell Signal #9803) and immunoprecipitated overnight at 4° C. After washing, the immunoprecipitates were run on an SDS-PAGE gel and blotted onto a PVDF membrane.

### 2.2 Results

2.2.1 Overexpression of Asb-a Renders PC12 Cells Insensitive to the NGF-Induced Block in Cell Proliferation

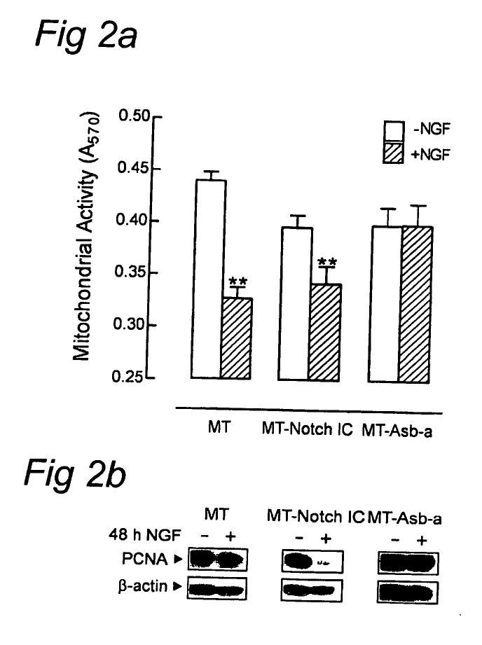

In Example 1 we demonstrated that Asb-a influences neuronal differentiation in vivo. Whether these effects are a direct consequence of Asb-a expression in the preneural population or are indirectly mediated via other cell types is unclear. Hence we investigated the effect of Asb-a gain-of-function during neuronal differentiation in vitro. To this end we used nerve growth factor (NGF)-treatment of pheochromocytoma cell lines (PC12), a well-established in vitro model for neuronal differentiation (Greene and Tischler, 1976; Vaudry et al., 2002). The most important characteristics of this model are the NGF-induced inhibition of cell proliferation followed by the extension of neurites by these cells. Importantly, PC12 cells overexpressing Asb-a seemed to continue proliferating upon NGF-induced differentiation, as they form overlaying colonies in contrast to control cells that remained in a monolayer. This notion was confirmed in an MTT assay (FIG. 2A), in which mitochondrial dehydrogenase activity was used as read out for cell number. After 3 days of culture, MT as well as MT-Notch IC transfected cells displayed markedly reduced proliferation when compared to non NGF-treated cells. However, PC12 cells transfected with MT-Asb-a were insensitive to NGF with respect to inhibition of cell proliferation, the mitochondrial activity was unchanged as compared to the non NGF-treated situation (FIG. 2A). In agreement, PCNA (proliferating cell nuclear antigen) protein expression, a marker for proliferation, was decreased in MT and MT-Notch IC transfected PC12 cells but not in MT-Asb-a transfected cells (FIG. 2B). Hence, Asb-a expression overcomes the NGF-induced block in cell proliferation in PC12 cells and thus Asb-a may directly influence cell fate in neuronal precursor cells by maintaining proliferation in these cells. In contrast Notch IC appears not to be able to maintain the proliferation of the PC12 cells.

2.2.2 Overexpression of Asb-a Blocks NGF-Induced Neurite Extension

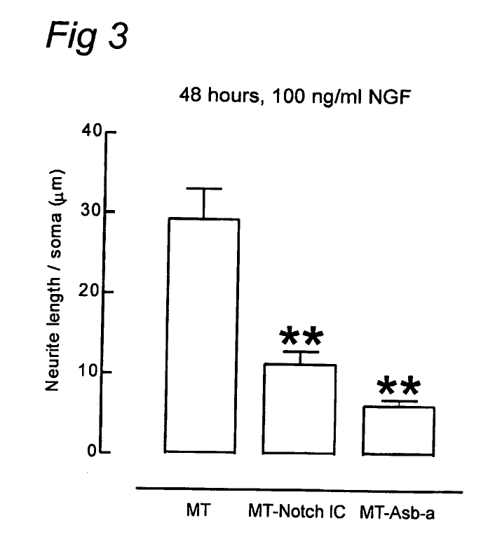

In order to investigate whether loss of NGF-induced inhibition of cell proliferation coincides with altered cell differentiation, we investigated the effects of Asb-a overexpression on NGF-induced neurite extension. To this end 10 pictures were taken at ×200 magnification (0.0325 mm²/picture) and both total neurite length and total number of somata were determined, blind to the experimental condition, in each microscope field with the use of an image analysis program (EFM Software, Rotterdam, The Netherlands). The mean neurite length per soma was taken as a relative measure of neuronal differentiation and we compared NGF-treated PC12 cells overexpressing Asb-a to cells transfected with a control (MT) vector and to cells overexpressing Notch1a IC. Notch IC impairs neurite extension in P19 and PC12 cells (Nye et al., 1994; Levy et al. 2002). As expected, control cells showed significant neurite outgrowth, which was markedly reduced by Notch1a IC overexpression, demonstrating that the strategy chosen is a valid approach for assessing neuronal differentiation in PC12 cells. Strikingly, Asb-a overexpression almost completely abolished any neurite outgrowth, superseding the effect of Notch1a IC (see FIG. 3). Thus the Asb-a-dependent insensitivity of PC12 cells to the NGF-induced block in proliferation coincides with the absence of NGF-induced terminal neuronal differentiation.

2.2.3 Overexpression of Asb-a Maintains Cells in the Neuronal Precursor Stage

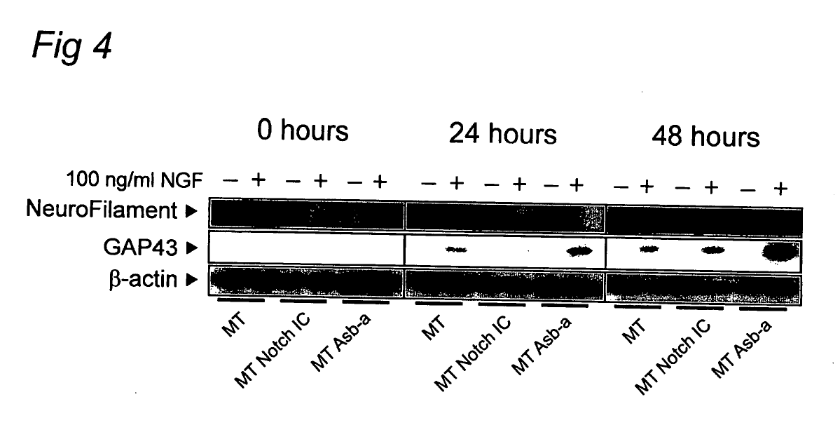

The lack of NGF-induced neurite extension in Asb-a-expressing PC12 cells may be caused by the incapacity of these cells to initiate neuronal differentiation but alternatively, may reflect a block further down in the execution of the neuronal differentiation program. To distinguish between these possibilities, we investigated the expression of GAP43, a marker for the neuronal precursor state (Karns et al., 1987; Esdar et al., 1999) and neurofilament, a terminal neuronal marker. As expected from the neurite extension experiments, transfection of MT-Asb-a and MT-Notch1a IC into PC12 cells inhibited neurofilament expression as assayed either by immunoblotting (FIG. 4) or immunohistochemistry (data not shown), thus confirming the neurite extension experiments. Importantly, however, Asb-a strongly enhanced the NGF-dependent expression of GAP43 (FIG. 4). We conclude that Asb-a overexpression does not lead to inhibition of the induction of neuronal differentiation per se, but blocks the transition from the proliferating neuronal precursor state to the post-mitotic terminally-differentiated phenotype, leading to an accumulation of neuronal precursors.

2.2.4 Asb-a Overexpression Affects the Transcriptional Activity of Specific Signal Transduction Pathways

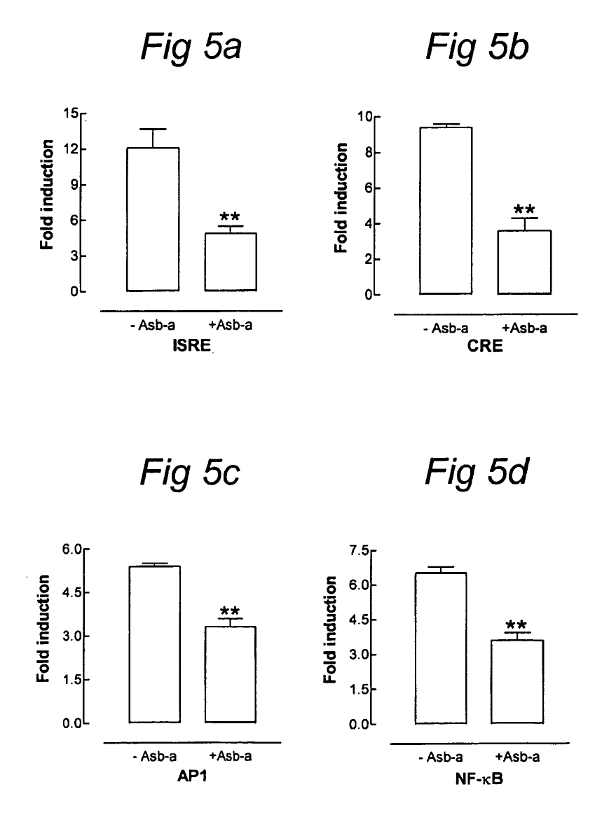

The remarkable effects of Asb-a on differentiation, both in vivo and in vitro, raise questions as to the molecular details by which these are achieved. Above we found Asb-a to be predominantly localised in the nucleus and as Asb-a belongs to the ankyrin repeats containing proteins that often function in transcriptional processes (Bork, 1993; Sedgwick and Smerdon, 1999), we hypothesised that Asb-a may participate in transcriptional regulation, especially as SOCS box containing proteins are well-established to downregulate cytokine and JAK/STAT-dependent transcriptional activation (Cooney, 2002; Kile and Alexander, 2001). Hence, we transfected Asb-a into HeLa cells together with reporter constructs containing the response element for various specific transcription factors and investigated the effects Asb-a expression on stimulus-induced transactivation (FIG. 5). As expected from a SOCS box containing protein, Asb-a overexpression strongly interfered with interferon-γ-dependent transactivation of ISRE (Interferon Stimulated Response Element) (FIG. 5A). Strikingly however, also the forskolin and Ca²⁺-dependent transactivation of the CRE (cAMP Response Elements) (FIG. 5B), c-fos/c-jun mediated transactivation of AP1 (FIG. 5C) and the TNF-dependent transactivation of κB enhancer containing reporters (FIG. 5D) were strongly downregulated by Asb-a expression. Thus these results show that Asb-a overexpression impairs transcriptional activation by specific signal transduction pathways.

2.2.5 Asb-a Overexpression Induced Stimulus-Dependent Ubiquitination of Signal Transduction Components

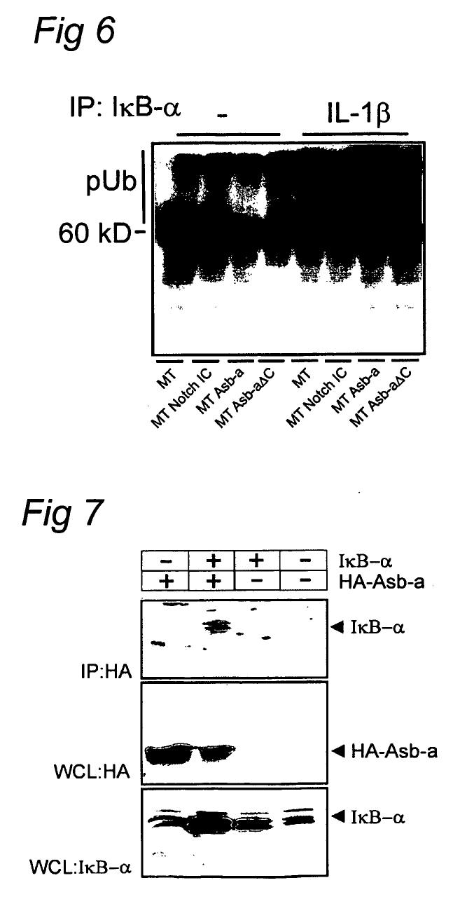

Based on the observations from the transactivation assay, we decided to further analyse the mechanism on how Asb-a represses these specific signalling cascades. The presence of a SOCS box in the Asb-a protein suggests that Asb-a might target proteins for ubiquitination, since SOCS box containing proteins can recruit proteins that belong to the family of E3 ubiquitin ligases. To determine whether Asb-a enhances the ubiquitination of putative target proteins, we overexpressed Asb-a in HeLa cells and stimulated these cells with 10 ng/ml IL-1β in order to activate the NF-κB signalling pathway, since this pathway is influenced by Asb-a in the reporter assays. Consistent with our hypothesis, we found that Asb-a overexpression led to an enhanced ubiquitination of IκB-α and p65 immunoprecipitates upon stimulation with IL-1β (FIG. 6, 7). A carboxy-terminal truncated version of Asb-a, Asb-aΔC, lacking the SOCS box (see Example 1), did not result in increased presence of ubiquitinated proteins demonstrating that the presence of the SOCS box was required for ubiquitination (FIG. 6, 7). Binding assays on Asb-a have already shown that Asb-a forms homodimers and heterodimerises with Notch IC and Nrarp (see above). As this interaction was not dependent on the SOCS box, but most likely occurred via the ankyrin repeats present in Asb-a as well as in Notch IC and Nrarp, we tested whether Asb-a associates with IκB-α, which possesses ankyrin repeats as well (Haskill et al., 1991). To this end, we transfected HA-tagged Asb-a (HA-Asb-a) in HEK293 cells that normally contain endogenous IκB-α Following immunoprecipitation of HA-Asb-a, we detected IκB-α after immunoblotting (data not shown). These results show that Asb-a can interact with IκBα. Thus, from our data we conclude that Asb-a enhances ubiquitination of proteins upon Il-1β stimulation and that these proteins are immunoprecipitated with IκBα and NF-κB/p65.

TABLE 1
The effects of asb-a and asb-aΔC overexpression
on her1, myoD and islet-1

| | | | asb-a | |
|---|---|---|---|---|
| | asb-a | | deltaC | |
| Her1 | (n = 62) | % | (n = 61) | % |
| Wildtype | 20 | 32.3 | 13 | 17.8 |
| diffuse expression | 7 | 11.3 | 20 | 27.4 |
| expression driven | 13 | 21 | 24 | 32.9 |
| to midline | | | | |
| reduced expression | 22 | 35.5 | 16 | 21.9 |

| | | | asb-a | |
|---|---|---|---|---|
| | asb-a | | deltaC | |
| MyoD | (n = 82) | % | (n = 113) | % |
| Wildtype | 15 | 18.3 | 20 | 17.7 |
| bended expression, | 34 | 41.5 | 49 | 43.4 |
| somitic expression | | | | |
| absent | | | | |
| somitic expression | 14 | 17.1 | 25 | 22.1 |
| reduced/absent | | | | |
| adaxial + somitic | 19 | 23.2 | 19 | 16.8 |
| expression reduced | | | | |

| | | | asb-a | |
|---|---|---|---|---|
| | asb-a | | deltaC | |
| Islet-1 | (n = 66) | % | (n = 44) | % |
| Wildtype | 13 | 19.7 | 14 | 34.1 |
| lateral neurons | 50 | 75.8 | 24 | 58.5 |
| shifted/reduced/absent | | | | |
| motorneurons absent | 1 | 1.52 | 3 | 7.32 |
| displacement of | 2 | 3.03 | 3 | 7.32 |
| cranial ganglia | | | | |

[image]

[image]

### REFERENCES

Anderson et al., 2001, Curr. Opin. Genet. Dev., 11(5), 554-560.
Appel, et al., 2001, BMC Dev. Biol. 1, 13.
Appel and Eisen, 1998, Development 125, 371-380.
Artavanis-Tsakonas et al., 1999, Science 284, 770-776.
Artinger et al., 1999, Development 126, 3969-3979.
Baker, 2000, Bioessays. 22:264-73.
Bierkamp and Campos-Ortega, 1993, Mech. Dev. 43, 87-100.
Blader et al., 1997, Development 124, 4557-4569.
Bork, 1993, Proteins 17, 363-374.
Chomczynskie and Sacchi, 1987, Anal Biochem. 162:156-9.
Cole and Ross, 2001, Dev Biol. 240:123-42.
Cooney, 2002, Shock 17, 83-90.
Cornell et al., 1999, Genetics 152, 567-576.
Cornell et al., 2002, Development 129, 2639-2648.
D'Amico et al., 2000, J. Biol. Chem. 275(42), 32649-57.
De Sepulveda et al., 2002, J. Biol. Chem. 275, 14005-14008.
Deblandre et al., 2001, Dev. Cell 1, 795-806.
Domen and Weissman 1999, Mol. Med. Today 5, 201-8.
Dornseifer et al., 1997, Mech. Dev. 63, 159-171.
Endo et al., 1997, Nature 387, 921-924.
Esdar et al., 1999, Eur. J. Neurosci. 11, 503-516.
Furutani-Seiki et al., 1996, Development 123, 229-239.
Granato et al., 1996, Development 123, 399-413.
Greene and Tischler, 1976, Proc. Natl. Acad. Sci. U.S.A. 73, 2424-2428.
Guibal et al., 2002, J. Biol. Chem. 277(1):218-24.
Haddon et al., 1998, Development 125, 359-370.
Haddon et al., 1998, Development 125, 4637-4644.
Haskill et al., 1991, Cell 65, 1281-1289.
Hilton et al., 1998, Proc. Natl. Acad. Sci. U.S.A. 95, 114-119.
Holley et al., 2002, Development 129, 1175-1183.
Inoue et al., 1994, Dev. Dyn. 199, 1-11.
Jiang et al., 2000, Nature 408, 475-479.
Joore et al., 1994, Mech Dev. 46:137-50
Karns et al., 1987, Science, 236, 597-600.
Kamizono et al., 2001, J. Biol. Chem. 276 (16), 12530-12538.
Kamura et al., 1998, Genes Dev. 12, 3872-3881.
Kidd et al., 1998, Genes. Dev. 12, 3728-3740.
Kile et al., 2000, Gene 258, 31-41.
Kile et al., 2001, Cell Mol. Life Sci. 58, 1627-1635.
Kile et al., 2002, Trends Biochem, Sci 27, 235-241.
Kim et al., 1997, Neurosci. Lett. 239, 113-116.
Kimmel et al., 1995 Dev. Dyn. 203, 253-310.
Knust, E. & Campos-Ortega, J. A. 1989, Bioessays 11, 95-100.
Kohroki et al., 2001, FEBS Lett. 505(2): 223-8.
Kortscbak et al., 2001, Dev. Genes Evol. 211, 350-354.
Korzh et al., 1993, Development 118, 417-425.
Korzh et al., 1998, Dev. Dyn. 213, 92-104.
Krebs et al., 2001, Dev. Biol. 238, 110-119.
Lahaye et al., 2002, Mech. Dev. 110, 113-124.
Lai et al, 2001, Dev. Cell 1, 783-794.
Lamar et al., 2001, Genes Dev. 15, 1885-1899.
Lecourtois and Schweisguth, 1998, Curr. Biol. 8, 771-774.
Levy et al., 2002, Dev. Neurosci., 24(1), 79-88.
Lieber et al., 1993, Genes Dev. 7, 1949-1965.
Lux et al., 1990, Nature 344, 36-42.
Matsumota et al., 1997, Blood 89, 3148-3154.
Matsuno et al., 1997, Development 124, 4265-4273.
Miele, L. & Osborne, B. 1999, Cell Physiol. 181, 393-409.
Morrison et al., 1999, Cell 96, 737-749.
Morrison et al., 1997, Cell 88, 287-298.
Mumm, J.S. & Kopan, R. 2000, Dev. Biol. 228, 151-165.
Naka et al., 1997, Nature 387, 924-929.
Nasevicius, A. & Ekker, S. C. 2000, Nat. Genet. 26, 216-220.
Nye et al., 1994, Development 120, 2421-2430.
Pourquie, O. 2000, Curr. Opin. Cell Biol. 12, 747-751.
Reinhard et al., 1994, Development 120:1767-75.
Schöler et al., 1989, EMBO J. 8, 2551-2557.
Schuldt and Brand, 1999, Dev. Biol. 205, 287-295.
Sedgwick, S. G. & Smerdon, S. J. 1999, Trends. Biochem. Sci. 24, 311-316.
Starr et al., 1997, Nature 387, 917-921.
Struhl et al., 1993, Cell 74, 331-345.
Takke et al., 1999, Development 126, 3005-3014.
Vaudry et al., 2002, Science, 296(5573), 1648-1649.
Van Eeden et al., 1996, Development 123, 153-164.
Verbeek, F. J. and Boon, P. J., 2002, IX. Proc. SPIE 4621, 65-76.
Weinberg et al., 1996, Development 122, 271-280.10, 363-369.
Weinmaster, 2000, Curr. Opin. Genet. Dev.
Westin, J. and Lardelli, M. 1997, Dev. Genes Evol. 207, 51-63.
Yoshimura et al., 1995, EMBO J. 14, 2816-2826.
Zhang et al., 1999, Proc. Natl. Acad. Sci. U.S.A. 96, 2071-2076.
Zhang et al., 2001, Proc. Natl. Acad. Sci U.S.A., 98(23), 13261-13275.
Ziegeler and Kanz, 1998 Curr. Opin. Hematol. 5, 434-40.

1. A method for in vitro expansion of a population of mammalian stem or progenitor cells, the method comprising the steps of:
(a) providing an effective amount of Asb-a polypeptide to stem or progenitor cells so that the intracellular concentration of the Asb-a polypeptide in said cells is sufficient to prevent differentiation of the cells; and,
(b) culturing the stem or progenitor cells for a period of time sufficient for the cells to divide and self-renew.

2. A method according to claim 1, wherein the intracellular concentration of the Asb-a polypeptide is maintained at a level sufficient to prevent differentiation of the cells for a period of time sufficient for the cells to divide and self-renew until the population has reached a predetermined size.

3. A method according to claim 1, whereby the Asb-a polypeptide is provided to the cells by addition of an exogenous Asb-a polypeptide to culture medium in which the cells are cultured.

4. A method according to claim 3, whereby the Asb-a polypeptide is fused to a transport moiety.

5. A method according to claim 4, whereby the Asb-a polypeptide is genetically fused to the transport moiety.

6. A method according to claim 4, wherein the transport moiety is a fragment of an HIV tat protein.

7. A method according to claim 1, wherein the Asb-a polypeptide is provided to the cells by introducing into the cells an exogenous nucleic acid comprising a nucleotide sequence encoding the Asb-a polypeptide.

8. A method according to claim 7, wherein the nucleic acid is an RNA molecule that is translated in the cells.

9. A method according to claim 7, wherein the exogenous nucleic acid is an expression vector in which the nucleotide sequence encoding the Asb-a polypeptide is operably linked to a promoter that is capable of regulating transcription of said polypeptide in the cells.

10. A method according to claim 9, wherein the expression vector is one that results in transient expression of the nucleotide sequence.

11. A method according to claim 10, wherein the vector is an episomal vector that does not replicate in the cells.

12. A method according to claim 9, wherein the vector comprises sites for recombination on either side flanking the coding sequence.

13. A method according to claim 12, wherein the vector integrates in to the genome of the cells.

14. A method according to claim 12, wherein the vector is a retroviral vector.

15. A method according to claim 1, wherein the stem or progenitor cells are hematopoietic stem cells, neural crest stem cells, mesenchymal stem cells, embryonic stem cells, endodermal stem cells, ectodermal stem cells, trophoblastic stem cells, mesodermal stem cells, cardiomyoblastic stem cells, endocrine stem cells, neurogenic precursor cells, skin precursor cells, renal precursor cells, hepatic precursor cells, pancreatic precursor cells or endothelial cells.

16. A method according to claim 15, wherein the stem or progenitor cells are human stem or progenitor cells.

17. An Asb-a polypeptide having an amino acid sequence with at least 39% identity with SEQ ID NO: 1 or 3, and wich is able to suppress NGF-induced terminal neuronal differentiation of PC12 cells, while allowing
(i) conversion of the PC12 cells to a neuronal precursor state and
(ii) proliferation of the PC-12 cells.

18. An isolated nucleic acid molecule comprising a nucleotide sequence that encodes an Asb-a polypeptide which is able to suppress NGF-induced terminal neuronal differentiation of PC12 cells, while allowing (i) conversion of the PC12 cells to a neuronal precursor state; and proliferation of the PC12 cells, which nucleotide sequence is selected from the group consisting of:
(a) a nucleotide sequence encoding a polypeptide comprising an amino acid sequence having at least 39% identity with the amino acid sequence of SEQ ID NO: 1 or 3;
(b) a nucleotide sequence that has at least 35% identity with a nucleotide sequence of SEQ ID NO:2 or 4;
(c) a nucleotide sequence, the complementary strand of which hybridizes to a nucleotide sequence acid having a sequence SEQ ID NO:2 or 4 under moderate or stringent conditions, and;
(d) a nucleotide sequence which differs from the nucleotide sequence of (c) by differences that do not result in amino acid sequence changes in an encoded polypeptide.

19. A vector comprising the nucleic acid molecule of claim 18.

20. An expression vector which comprises the vector of claim 18, and further comprises an operably linked promoter capable of driving expression of the coding sequence in a host cell into which the vector is introduced.

21. An expression vector according to claim 20, wherein the promoter is active in stem or progenitor cells.

22. An expression vector according to claim 21, wherein the promoter is selected from the group consisting of an Oct4 promoter, an Oct5 promoter, a TCF-regulated promoter, a LIF-regulated promoter, and a Notch IC/herl targeted promoter.

23. A host cell comprising a vector according to claim 19.

24. A method for producing an Asb-a polypeptide, comprising the step of culturing the host cell of claim 29 under conditions conducive to the expression of the polypeptide, and, optionally recovering the polypeptide.

25. A host cell according to claim 29, which is a stem or progenitor cell.

26. A stem or progenitor cell comprising
(i) an exogenous Asb-a polypeptide,
(ii) an exogenous nucleotide sequence encoding an Asb-a polypeptide, or
(iii) both (i) and (ii).

27. A stem or progenitor cell according to claim 26, which is a hematopoietic stem cell, a neural crest stem cell, a mesenchymal stem cell, an embryonic stem cell, an endodermal stem cell, an ectodermal stem cell, a trophoblastic stem cell, a mesodermal c stem cell, a cardiomyoblastic stem cell, an endocrine stem cell, a neurogenic precursor cell, a skin precursor cell, a renal precursor cell, a hepatic precursor cell, a pancreatic precursor cell or an endothelial cell.

28. A pharmaceutical composition comprising the stem or progenitor cell of claim 26 and a pharmaceutically acceptable diluent or carrier.

29. A host cell comprising an expression vector according to claim 20.
